(19) United States
(12) Patent Application Publication
(10) Pub. No.: US 2026/0356826 A1
(43) Pub. Date: Nov. 12, 2026

(54) SALTS OF AMINO QUINAZOLINE DERIVATIVES

(71) Applicant: CHIESI FARMACEUTICI S.P.A., Parma (IT)

(72) Inventors: Francesco AMADEI, Parma (IT); Davide DE ANGELIS, Parma (IT); Irene BASSANETTI, Parma (IT); Susan Elizabeth OSBOURN, Parma (IT); Andrew David CARR, Parma (IT); Massimiliano MARI, Parma (IT)

(73) Assignee: CHIESI FARMACEUTICI S.P.A., Parma (IT)

(21) Appl. No.: 19/486,109

(22) PCT Filed: May 21, 2024
(86) PCT No.: PCT/EP2024/063878

§ 371 Date: Nov. 20, 2025

(30) Foreign Application Priority Data
May 22, 2023 (EP) 23174497.0

(51) Int. Cl. C07D 413/14; A61K 31/517

(52) U.S. Cl. CPC C07D 413/14; A61K 31/517

(57) ABSTRACT

The invention relates to salts derivatives of (R)-6-(5-fluoropyridin-2-yl)-8-methoxy-N-(1-(5-methyl-1,2,4-oxadiazol-3-yl)ethyl)quinazolin-4-amine, particularly the invention relates to mesylate and esylate salts of (R)-6-(5-fluoropyridin-2-yl)-8-methoxy-N-(1-(5-methyl-1,2,4-oxadiazol-3-yl)ethyl)quinazolin-4-amine, pharmaceutical compositions containing them and therapeutic use thereof. The salts of the invention may be useful in the treatment of diseases or conditions associated with respiratory diseases, in particular chronic cough.

### FIELD OF THE INVENTION

The present invention relates to salts of P2X purinoceptor 3 (hereinafter P2X₃) inhibitors; particularly the invention relates to mesylate and esylate salts of (R)-6-(5-fluoropyridin-2-yl)-8-methoxy-N-(1-(5-methyl-1,2,4-oxadiazol-3-yl)ethyl)quinazolin-4-amine, pharmaceutical compositions containing them and therapeutic use thereof.

The mesylate and esylate salts of the invention may be useful in the treatment of many disorders associated with P2X₃ receptor mechanisms, such as respiratory diseases including cough, asthma, idiopathic pulmonary fibrosis (IPF) and chronic obstructive pulmonary disease (COPD).

### BACKGROUND OF THE INVENTION

P2X receptors are cell surface ion channels activated by extracellular Adenosine 5-TriPhosphate (ATP). P2X receptor family are trimeric assemblies composed of seven distinct subunit subtypes (P2X₁₋₇) that assemble as homomeric and heteromeric channels. Homomeric P2X₁, P2X₂, P2X₃, P2X₄, P2X₅, and P2X₇ channels and heteromeric P2X_{2/3} and P2X_{1/5} channels have been fully characterized following heterologous expression. All P2X receptors are non-selective cation channels permeable to Na+ and Ca+ ions and are activated by ATP; however, the pharmacology of the receptor subtypes varies with respect to sensitivity to ATP and to small molecules antagonists. (K Kaczmarek-Hajek et al: Molecular and functional properties of P2X receptors—recent progress and persisting challenges; Purinergic Signalling 8:375-417, 2012).

ATP enhances citric acid-evoked and histamine-evoked cough in preclinical models, wherein such effects can be attenuated by P2X₃ selective antagonists (Kamei J and Takahashi Y: Involvement of ionotropic purinergic receptors in the histamine-induced enhancement of the cough reflex sensitivity in guinea pigs, Eur J Pharmacol. 2006 Oct. 10; 547(1-3):160-4). In humans, local delivery of ATP initiates cough and bronchospasm (Basoglu O K et al: Effects of aerosolized adenosine 5′-triphosphate vs adenosine 5′-monophosphate on dyspnea and airway caliber in healthy non-smokers and patients with asthma, Chest. October; 128(4):1905-9, 2005).

The therapeutic promise of P2X₃ antagonists for the treatment of chronic cough was first recognized by Ford and Undem (Ford A P, Undem B J: The therapeutic promise of ATP antagonism at P2X₃ receptors in respiratory and urological disorders, Front Cell Neurosci, December 19; 7:267, 2013). P2X₃ are expressed by airway afferent nerves and mediate hypersensitivity of the cough reflex, which is dramatically reduced by the oral P2X₃ antagonist, AF-219 (Abdulgawi et al: P2X₃ receptor antagonist (AF-219) in refractory chronic cough: a randomised, double-blind, placebo-controlled phase 2 study, Lancet 385, 1198-205, 2015).

Several compounds have been described in the literature as P2X₃ inhibitors.

WO2020239951 A1 (Chiesi Farmaceutici S.P.A.) discloses amino quinazoline derivatives and the use of amino quinazoline derivatives as P2X₃ inhibitors for the treatment of disorders associated with P2X₃ receptor mechanisms, such as respiratory diseases including cough, asthma, idiopathic pulmonary fibrosis (IPF) and chronic obstructive pulmonary disease (COPD). In particular, (R)-6-(5-fluoropyridin-2-yl)-8-methoxy-N-(1-(5-methyl-1,2,4-oxadiazol-3-yl)ethyl) quinazolin-4-amine is disclosed as a potent and selective P2X₃ inhibitor.

Although several P2X₃ inhibitors have been disclosed so far as above reported, there is still a need for further P2X₃ inhibitors.

The identification of further P2X₃ inhibitors, endowed with an improved solubility, stability and suitable to be formulated for the oral route, would be particularly advantageous.

The present invention addresses the above mentioned needs by providing the compound of the invention, (R)-6-(5-fluoropyridin-2-yl)-8-methoxy-N-(1-(5-methyl-1,2,4-oxadiazol-3-yl)ethyl)quinazolin-4-amine mesylate and esylate salts.

### SUMMARY OF THE INVENTION

The present invention relates to a salt of (R)-6-(5-fluoropyridin-2-yl)-8-methoxy-N-(1-(5-methyl-1,2,4-oxadiazol-3-yl)ethyl)quinazolin-4-amine (compound A, shown below) selected from mesylate salt and esylate salt.

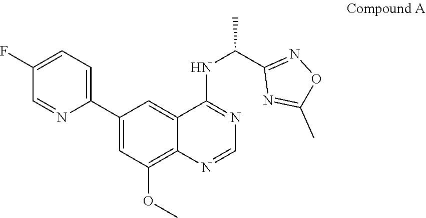 [SMILES: COc1cc(-c2ccc(F)cn2)cc2c(N[C@H](C)c3noc(C)n3)ncnc12]

In one aspect, the present invention relates to a crystalline mesylate salt Form 2 of Compound A, wherein the crystalline Form 2 is characterized by an X-ray powder diffraction pattern comprising characteristic peaks at 6.6, 11.8 and 19.2±0.2 degrees 2-theta.

In a second aspect, the invention relates to a crystalline esylate salt Form 1 of Compound A, wherein the crystalline Form 1 is characterized by an X-ray powder diffraction pattern comprising characteristic peaks at 5.8, 16.3 and 17.7±0.2 degrees 2-theta.

In a third aspect, the present invention relates to a pharmaceutical composition comprising the mesylate salt or esylate salt of compound A, either alone or in combination with another one or more active ingredient, in admixture with one or more pharmaceutically acceptable carrier or excipient.

In a further aspect the present invention relates to a pharmaceutical composition comprising the mesylate salt or esylate salt of compound A according to the above aspects for use as a medicament.

In a further aspect the present invention relates to the mesylate salt or esylate salt of compound A or a pharmaceutical composition according to the above aspects for use in the treatment of any disease wherein the P2X₃ receptors are involved.

In a further aspect, the present invention relates to the mesylate salt or esylate salt of compound A or a pharmaceutical composition as above described for use in the prevention and/or treatment of respiratory diseases including cough, sub-acute or chronic cough, treatment-resistant cough, idiopathic chronic cough, post-viral cough, iatrogenic cough, asthma, idiopathic pulmonary fibrosis (IPF), chronic obstructive pulmonary disease (COPD) and cough associated with respiratory diseases such as COPD, asthma and bronchospasm, preferably in the treatment of chronic cough.

In a further aspect, the invention relates to a process for the preparation of a crystalline salt of (R)-6-(5-fluoropyridin-2-yl)-8-methoxy-N-(1-(5-methyl-1,2,4-oxadiazol-3-yl)ethyl)quinazolin-4-amine (compound A) selected from mesylate and esylate salt, comprising the steps of:
1) suspending compound A free base in a polar solvent or solvent mixture;
2) adding methanesulfonic or ethanesulfonic acid; and
3) isolating the resulting crystalline salt by filtration.

### BRIEF DESCRIPTION OF THE DRAWINGS

FIG. 1 is a characteristic XRPD pattern for free base Form A of Compound A.

FIG. 2 is a DSC profile of free base Form A of Compound A.

FIG. 3 is a TGA profile of free base Form A of Compound A.

FIG. 4 is a DVS isotherm for free base Form A of Compound A.

FIG. 5 is a characteristic XRPD pattern for mesylate salt Form 2 of Compound A.

FIG. 6 is a DSC profile of mesylate salt Form 2 of Compound A.

FIG. 7 is a TGA profile of mesylate salt Form 2 of Compound A.

FIG. 8 is a DVS isotherm for mesylate salt Form 2 of Compound A.

FIG. 9 is a characteristic XRPD pattern for mesylate salt Form 3 of Compound A.

FIG. 10 is a characteristic XRPD pattern for mesylate salt Form 4 of Compound A.

FIG. 11 is a characteristic XRPD pattern for mesylate salt Form 6 of Compound A.

FIG. 12 is a characteristic XRPD pattern for esylate salt Form 1 of Compound A.

FIG. 13 is a DSC profile of esylate salt Form 1 of Compound A.

FIG. 14 is a TGA profile of esylate salt Form 1 of Compound A.

FIG. 15 is a variable temperature XRPD pattern of the transition from mesylate salt Form 2 of Compound A to mesylate salt Form 3 of Compound A.

FIG. 16 is an XRPD pattern of mesylate salt Form 2 of Compound A at zero weeks, two weeks, one month, two months, three months, six months and one year of storage in an open vial at 25 °C and 60% RH.

FIG. 17 is an XRPD pattern of mesylate salt Form 2 of Compound A at zero weeks, two weeks, one month, two months, three months, six months and one year of storage in a closed vial at 25 °C and 60% RH.

FIG. 18 is an XRPD pattern of mesylate salt Form 2 of Compound A at zero weeks, two weeks, one month, two months, three months, six months and one year of storage in an open vial at 40 °C and 75% RH.

FIG. 19 is an XRPD pattern of mesylate salt Form 2 of Compound A at zero weeks, two weeks, one month, two months, three months, six months and one year of storage in a closed vial at 40 °C and 75% RH.

FIG. 20 is an XRPD pattern of free base Form A of Compound A at 0, 28, 56 and 84 days of storage in an open vial at 40 °C and 75% RH.

FIG. 21 is an XRPD pattern of mesylate salt Form 2 of Compound A at 0, 28, 56 and 84 days of storage in an open vial at 40 °C and 75% RH.

FIG. 22 is an XRPD pattern of esylate salt Form 1 of Compound A at 0, 28, 56 and 84 days of storage in an open vial at 40 °C and 75% RH.

FIG. 23 is a dissolution profile of free base Form A of Compound A in FaSSIF, FeSSIF and FaSSGF.

FIG. 24 is a dissolution profile of mesylate salt Form 2 of Compound A in FaSSIF, FeSSIF and FaSSGF.

FIG. 25 is a two-sector dissolution profile of free base Form A of Compound A.

FIG. 26 is a two-sector dissolution profile of mesylate salt Form 2 of Compound A.

FIG. 27 is a pH-adjusted two-sector dissolution profile of free base Form A of Compound A and mesylate salt Form 2 of Compound A.

FIG. 28 is a dissolution profile of mesylate salt Form 2, esylate salt Form 1 and free base Form A compared with other salts of Compound A in FaSSGF

FIG. 29 is a pH-adjusted two-sector dissolution profile of esylate salt Form 1, free base Form A and mesylate salt Form 2 of Compound A.

### DETAILED DESCRIPTION OF THE INVENTION

### Definitions

Unless defined otherwise, all technical and scientific terms used herein have the same meanings as commonly understood by the skilled in the art.

The term “compound A” refers to (R)-6-(5-fluoropyridin-2-yl)-8-methoxy-N-(1-(5-methyl-1,2,4-oxadiazol-3-yl)ethyl)quinazolin-4-amine.

It is to be understood that besides the compound A showing a preferred (R) configuration, the racemic form and mixtures in other proportion with enantiomer (S) are encompassed within the scope of the present invention.

The term “mesylate” refers to any salt of methanesulfonic acid, wherein the salt has a stoichiometry of 1:1 (R)-6-(5-fluoropyridin-2-yl)-8-methoxy-N-(1-(5-methyl-1,2,4-oxadiazol-3-yl)ethyl)quinazolin-4-amine: mesylate.

The term “mesylate salt Form 2” refers to mesylate salt of Compound A Form 2.

The term “mesylate salt Form 3” refers to mesylate salt of Compound A Form 3.

The term “mesylate salt Form 4” refers to mesylate salt of Compound A Form 4.

The term “mesylate salt Form 6” refers to mesylate salt of Compound A Form 6.

The term “esylate” refers to any salt of ethanesulfonic acid, wherein the salt has a stoichiometry of 1:1 (R)-6-(5-fluoropyridin-2-yl)-8-methoxy-N-(1-(5-methyl-1,2,4-oxadiazol-3-yl)ethyl)quinazolin-4-amine:esylate.

The term “esylate salt Form 1” refers to esylate salt of Compound A Form 1.

The terms “polymorph”, “polymorphous form”, “crystalline”, “crystalline form” and “form” mean a compound having a particular molecular packing arrangement in a crystal lattice. X-ray powder diffraction (XRPD), proton nuclear magnetic resonance (NMR), differential scanning calorimetry (DSC), thermogravimetric analysis (TGA) and dynamic vapor sorption (DVS) are techniques that can be used for identifying and characterising properties of polymorphous forms, such as the polymorphous forms of the mesylate and esylate salts and the free base compound A as described herein.

The term “crystalline solid” refers to any polymorph according to the invention prior to isolation.

The term “XRPD” means X-ray powder diffraction. XRPD is a technique for providing an analytical characterisation of a sample. Polymorphous forms are characterized by having different XRPD patterns.

The term “characteristic peaks” means peaks observed in an XRPD pattern that are characteristic for a particular crystalline form differentiating it from other crystalline forms.

The term “representative peaks” means a selection of the characteristic peaks in an XRPD pattern for a particular form. The representative peaks correspond to the characteristic peaks having the strongest signal in the XRPD pattern. These peaks distinguish the particular crystalline form from other crystalline forms. In the present disclosure, characteristic and representative peaks values are reported as measured by X-ray powder diffraction using an X-ray wavelength of 1.5406 Å (copper radiation source, Cu Kα). In one embodiment, such peaks should be understood as comprising an error margin of ±0.2 degrees 2-theta. In a further embodiment, the error margin should be understood as being ±0.1 degrees 2-theta.

The term “NMR” means proton nuclear magnetic resonance. NMR spectra provide structural information about organic compounds based on energy absorption by hydrogen atoms.

The term “DSC” means differential scanning calorimetry. DSC is a thermoanalytical technique measuring the difference in the amount of heat required to increase the temperature of a test compound and a reference as a function of temperature. The output is a differential thermogram and can for instance be used to estimate melting temperatures of the test compound. It is used for characterising polymorphous forms of salts.

The term “TGA” means thermogravimetric analysis. TGA is a technique where the mass of a substance is monitored as a function of temperature as a sample is subjected to a controlled temperature program in a controlled atmosphere. It is used for characterising polymorphous forms of salts.

The term “DVS” means dynamic vapour sorption. DVS is a gravimetric sorption technique measuring a sample's absorption and desorption of a solvent, such as water vapour. It is used for characterising polymorphous forms of salts.

The term “LC/UV” means liquid chromatography coupled with ultraviolet detection.

The term “UPLC/UV/MS” means UltraPerformance Liquid Chromatography coupled with ultraviolet detection and mass spectrometry.

The term “amorphous” refers to a solid form that is not crystalline. An amorphous form does not have a particular packing arrangement of molecules of a compound in a crystal lattice. In the present application the compound in question is compound A.

The term “anhydrous” refers to a crystalline form not containing any water molecules among molecules of a compound packed in its crystal lattice.

The term “hemihydrate” refers to a crystalline hydrate containing one water molecule for every two molecules of a compound in its crystal lattice.

The term “polar solvent” refers to both protic and aprotic polar solvents, including organic solvents and water.

The term “solvent mixture” refers to a mixture of at least two solvents, in variable proportions.

The term “antisolvent” refers to a liquid that, when combined with a solution of Compound A, and for example methanesulfonic or ethanesulfonic acid, reduces the solubility of the respective salt in the solution causing its crystallization or precipitation, in some instances spontaneously, and in other instances with additional steps, such as seeding, cooling, scratching, and/or concentrating.

The term “FaSSGF” means Fasted State Simulated Gastric Fluid. FaSSGF is a dissolution medium used for studying the behaviour of orally administered drugs in the stomach after consumption of a glass of water. The osmolarity and average pH of FaSSGF is similar to that of fasted gastric fluid. Tests using FaSSGF can generate stability, solubility and dissolution data.

The term “FaSSIF” means Fasted State Simulated Intestinal Fluid. FaSSIF is a dissolution medium used for studying the behaviour of orally administered drugs in the upper intestine after consumption of a glass of water. The osmolarity and average pH of FaSSIF is similar to that of fasted intestinal fluid. The data from FaSSIF testing can be used for evaluating drug dissolution and potential absorption in upper intestine fluid.

The term “FeSSIF” means Fed State Simulated Intestinal Fluid. FeSSIF is a dissolution medium used for studying the behaviour of orally administered drugs in the upper intestine in a fed state. The osmolarity and average pH of FeSSIF is similar to that of fed intestinal fluid. The data from FeSSIF testing can be used for evaluating drug dissolution and potential absorption in upper intestine fluid after a meal.

The term “relative humidity”, abbreviated to RH, means the percentage of the amount of moisture present in the air compared to the amount of moisture upon saturation.

The term “room temperature”, abbreviated to RT, means a temperature in the range of about 15 °C to about 25 °C, with an average of about 23 °C.

The term “treating”, or “treatment” of a disease state includes: (i) inhibiting the disease state, i.e. arresting the development of the disease state or its clinical symptoms, or (ii) relieving the disease state, i.e. causing temporary or permanent regression of the disease state or its clinical symptoms.

The term “preventing”, or “prevention” of a disease state includes causing the clinical symptoms of the disease state not to develop in a subject that may be exposed to or predisposed to the disease state, but does not yet experience or display symptoms of the disease state. For example, treating or preventing a respiratory disease or disorder includes treating or preventing the symptoms the disorder such as cough and/or urge to cough associated with a respiratory disease.

The term “therapeutically effective amount” means an amount of a compound that, when administered to a subject for treating a disease state, is sufficient to affect such treatment for the disease state. The “therapeutically effective amount” will vary depending on the compound, disease state being treated, the severity or the disease treated, the age and relative health of the subject, the route, and form of administration.

The present invention relates to novel salts and crystalline forms of Compound A, an inhibitor of P2X₃ receptor. Compound A, (R)-6-(5-fluoropyridin-2-yl)-8-methoxy-N-(1-(5-methyl-1,2,4-oxadiazol-3-yl)ethyl)quinazolin-4-amine, having the chemical structure reported above, can be prepared by using procedures described in International Publication WO2020239951 A1.

The novel salts of Compound A, especially the mesylate salt and the esylate salt of compound A as described herein, can be provided stably and constantly from the standpoint of the manufacturing process and are useful in the treatment of P2X₃ mediated conditions.

In particular, the mesylate and the esylate salts crystalline forms have many unexpected properties as described in more detail below.

Advantageously, the mesylate salt and the esylate salt of compound A show a higher degree of solubility in simulated gastric fluid (FaSSGF) in comparison with the free base Form A. As can be appreciated in Table 8, Example 8, of the Experimental part, after 60 minutes the mesylate and esylate salts of compound A reach a concentration equal to or greater than about 25 mg/ml, higher than the free base Form A and surprisingly higher than other salts, such as the tosylate, the sulphate, the oxalate, saccharinate, fumarate and other salts of Compound A.

More advantageously, the mesylate salt of compound A, in particular the crystalline Form 2, surprisingly shows a potentially improved bioavailability demonstrated by the supersaturation duration. As can be appreciated in Example 10, the mesylate salt Form 2 of compound A was tested in a two-sector model to evaluate the “spring-parachute effect”. The term “spring-parachute effect” or, more simply, “parachute effect” refers to the high solubility observed in gastric fluid (spring) followed by supersaturation (parachute) in the intestinal fluid. The longer the duration of the supersaturation, the higher the potential bioavailability of the compound, as this phenomena predicts an higher dissolved amount available for absorption for a longer time.

Surprisingly, the mesylate salt Form 2 of compound A shows a longer duration of the parachute effect than the free base Form A, with an estimated supersaturation duration of 14 minutes versus 8 minutes shown by the free base Form A.

As shown in Example 11, the mesylate shows better formulability in solution than free base Form A which tends to give suspensions or gels, even if in presence of different excipients like solubilizing agents, surfactants and/or hydrophilic polymers which can promote supersaturation effect.

The longer supersaturation duration is confirmed even when the mesylate salt Form 2 of compound A is formulated in combination with excipients, such as HPMC, as shown in Example 12.

As can be appreciated in Table 15, the formulation B comprising the mesylate salt Form 2 shows a supersaturation measured at 31 minutes against the 16 minutes of the formulation A comprising the free base Form A, clearly predicting an advantageous increased in vivo bioavailability, particularly useful for the formulation for oral administration.

As further advantage, the mesylate salt of compound A is stable for several months, during both regular and stressed storage conditions, as shown in Example 7.

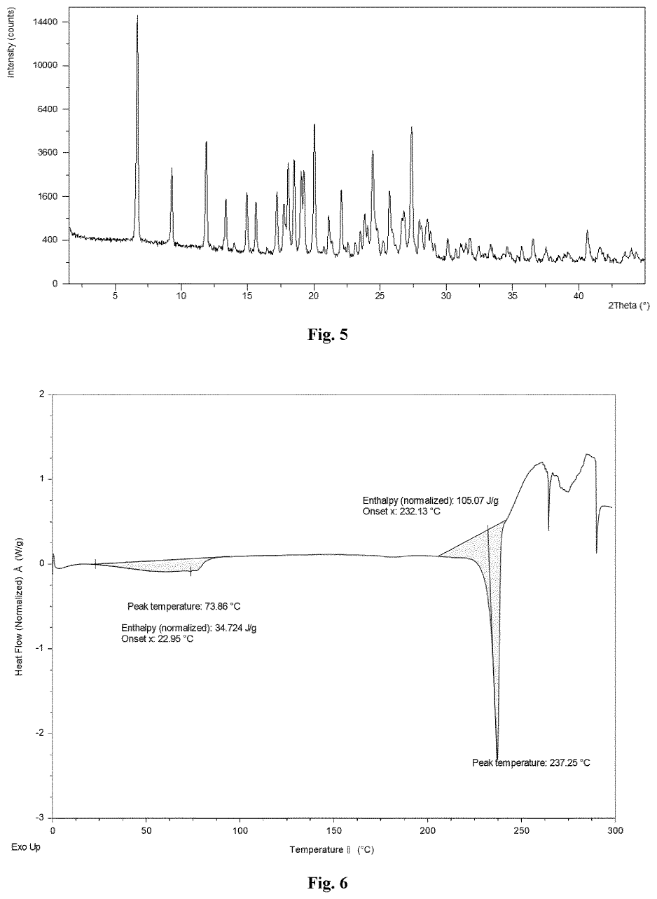

The mesylate salt of compound A exists in one or more crystalline forms. The identified crystalline polymorphs are designated mesylate salt Form 2, Form 3, Form 4 and Form 6. In one aspect, the invention provides a mesylate salt of compound A in a crystalline Form 2. The XRPD pattern of the mesylate salt Form 2 of compound A is substantially as shown in FIG. 5.

In one preferred embodiment, the XRPD pattern of mesylate salt Form 2 has representative diffraction peaks at diffraction angles 2-theta corresponding to 6.6, 11.8 and 19.2±0.2 degrees. In a more preferred embodiment, the XRPD pattern of mesylate salt Form 2 has representative diffraction peaks at diffraction angles 2-theta corresponding to 6.6, 9.3, 11.8, 19.2 and 27.4±0.2 degrees. In an even more preferred embodiment, the XRPD pattern of mesylate salt Form 2 has characteristic diffraction peaks at diffraction angles 2-theta corresponding to 6.6, 9.3, 11.8, 18.1, 18.5, 19.2, 20.0, 22.4, 25.7 and 27.4±0.2 degrees.

In one embodiment, the mesylate salt Form 2 was analysed by NMR, as reported in Example 2.

In one embodiment, the mesylate salt Form 2 was analysed by DSC. Mesylate salt Form 2 has a DSC profile substantially as shown in FIG. 6. Preferably the mesylate Form 2 has a differential scanning calorimetry (DSC) onset melting temperature in the range 220-240 °C, more preferably about 232.13 °C.

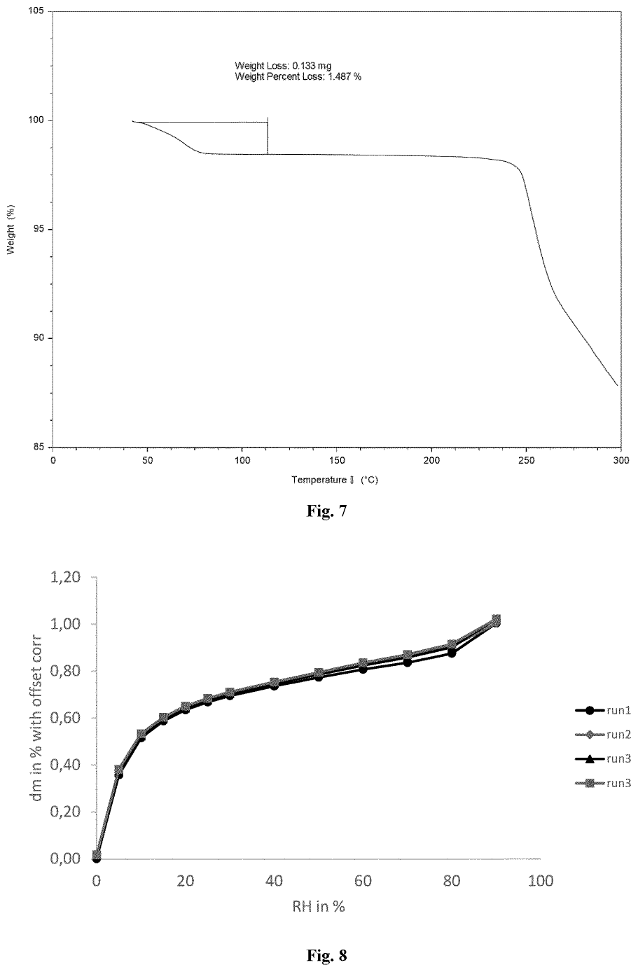

In one embodiment, the mesylate salt Form 2 was analysed by thermogravimetric analysis (TGA). Mesylate salt Form 2 has a TGA profile substantially as shown in FIG. 7. In one embodiment, mesylate salt Form 2 is characterized by a 1.487% weight loss by TGA in the range 30-120 °C, indicating that mesylate salt Form 2 is hemihydrate. As hydrate, mesylate salt exists in only one stable form (hemihydrate) differently to unstable hydrate forms of free base Compound A. Thus, in a preferred embodiment, the present invention provides a crystalline salt of compound A which is the crystalline mesylate salt hemihydrate form.

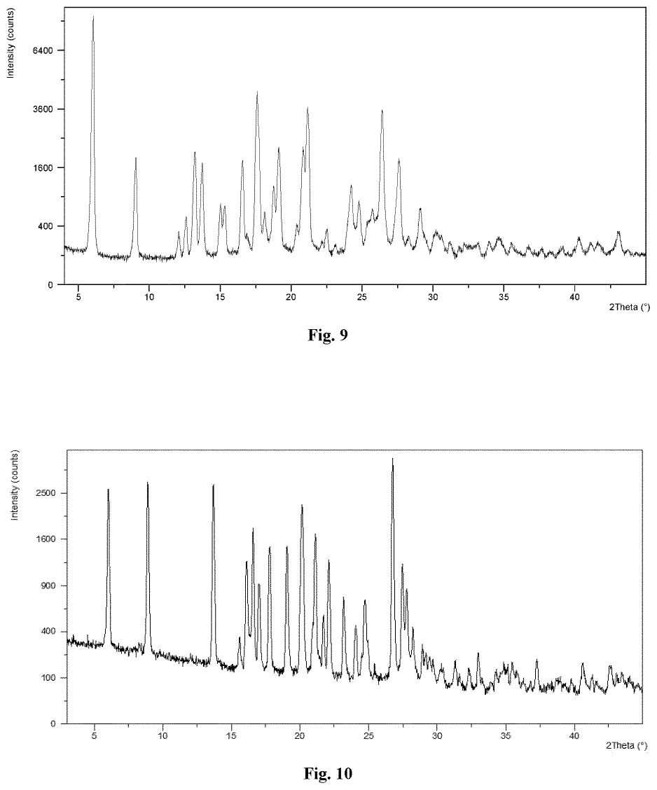

In one embodiment, the mesylate Form 2 was characterized by dynamic water sorption (DVS) analysis. Mesylate salt Form 2 has a DVS profile substantially as shown in FIG. 8. Hygroscopicity was noted between 0% and 90% RH, with the sample gaining a total of about 1.049 wt % which does not correspond to a complete dehydration/hydration of the solid (slow kinetic process). Approximately half of the weight gain occurred between 0% and 15% RH, confirming the ease of water uptake of instable anhydrous form. The adsorption and desorption profiles are similar, although with some hysteresis between 40% and 80% RH.

Mesylate salt Form 2 of compound A may be prepared for instance as described in Example 2 methods A, B, C and D.

In a further aspect, the invention provides a mesylate salt of compound A in a crystalline Form 3. The XRPD pattern of the mesylate salt Form 3 of compound A is substantially as shown in FIG. 9.

In one embodiment, the XRPD pattern of mesylate salt Form 3 has representative diffraction peaks at diffraction angles 2-theta corresponding to 6.0, 17.6 and 26.4±0.2 degrees. In one embodiment, the XRPD pattern of Form 3 has representative diffraction peaks at diffraction angles 2-theta corresponding to 6.0, 9.0, 17.6, 21.2 and 26.4±0.2 degrees. In one embodiment, the XRPD pattern of Form 3 has characteristic diffraction peaks at diffraction angles 2-theta corresponding 6.0, 9.0, 13.2, 13.7, 16.6, 17.6, 19.1, 21.2, 26.4 and 27.6±0.2 degrees.

The mesylate salt Form 3 was analysed by DSC. Preferably, the mesylate Form 3 has a differential scanning calorimetry (DSC) onset melting temperature in the range 220-240 °C, more preferably about 229.65 °C.

The mesylate salt Form 3 was analysed by TGA. Negligible weight loss was observed until about 237.5 °C, indicating that mesylate salt Form 3 is anhydrous.

In a further aspect, the invention provides a mesylate salt of compound A in a crystalline Form 4. The XRPD pattern of the mesylate salt Form 4 of compound A is substantially as shown in FIG. 10. In one embodiment, the XRPD pattern of mesylate salt Form 4 has representative diffraction peaks at diffraction angles 2-theta corresponding to 6.0, 8.8 and 17.7±0.2 degrees. In one embodiment, the XRPD pattern of mesylate salt Form 4 has representative diffraction peaks at diffraction angles 2-theta corresponding to 6.0, 8.8, 17.7, 21.1 and 26.7±0.2 degrees. In one embodiment, the XRPD pattern of mesylate salt Form 4 has characteristic diffraction peaks at diffraction angles 2-theta corresponding 6.0, 8.8, 13.6, 16.5, 17.7, 18.9, 21.1, 22.0, 24.7 and 26.7±0.2 degrees.

The mesylate salt Form 4 was analysed by DSC. Preferably the mesylate Form 4 has a DSC onset melting temperature in the range 220-240 °C, more preferably about 236.74 °C.

The mesylate salt Form 4 was analysed by TGA. Negligible weight loss was observed until about 240 °C, indicating that mesylate salt Form 4 is anhydrous.

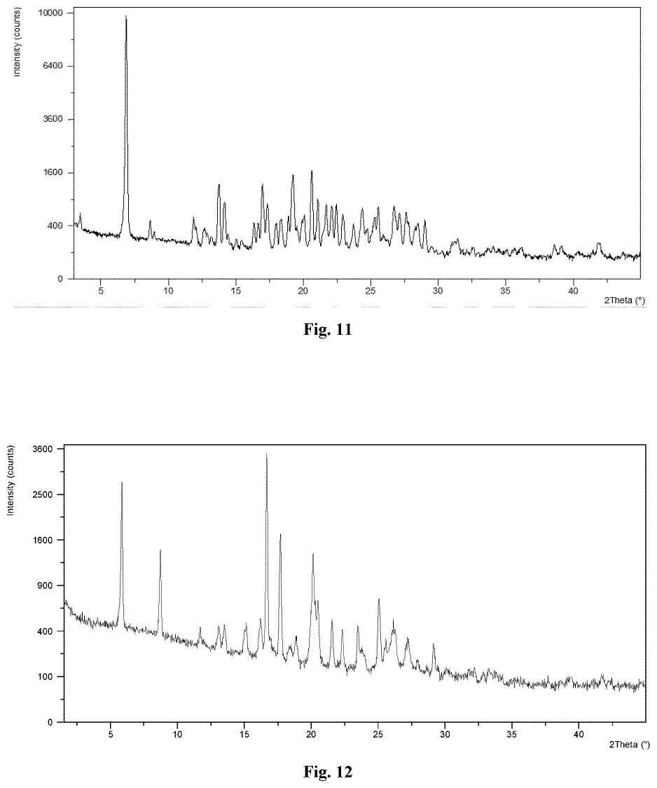

In a further aspect, the invention provides a mesylate salt of compound A in a crystalline Form 6. The XRPD pattern of the mesylate salt Form 6 of compound A is substantially as shown in FIG. 11. In one embodiment, the XRPD pattern of Form 6 has representative diffraction peaks at diffraction angles 2-theta corresponding to 6.8, 8.5 and 19.2±0.2 degrees. In one embodiment, the XRPD pattern of Form 6 has representative diffraction peaks at diffraction angles 2-theta corresponding to 6.8, 8.5, 11.8, 14.1 and 19.2±0.2 degrees. In one embodiment, the XRPD pattern of Form 6 has characteristic diffraction peaks at diffraction angles 2-theta corresponding to 6.8, 8.5, 11.8, 14.1, 16.9, 17.2, 19.2, 21.0, 24.3 and 25.4±0.2 degrees.

The mesylate salt Form 6 was analysed by DSC. Preferably the mesylate salt Form 6 has a DSC onset melting temperature in the range 220-240 °C, more preferably about 235.43 °C.

The mesylate salt Form 6 was analysed by TGA. Negligible weight loss was observed until about 237.5 °C, indicating that the mesylate salt Form 6 is anhydrous.

The mesylate salt Form 6 was analysed by DVS. No hygroscopicity was noted as a total of 0.3% water uptake was registered between 20% and 80% RH.

In another aspect, the invention provides the esylate salt of compound A as described herein. The XRPD pattern of the esylate salt Form 1 of compound A is substantially as shown in FIG. 12. In one embodiment, the XRPD pattern of esylate salt Form 1 has representative diffraction peaks at diffraction angles 2-theta corresponding to 5.8, 16.3 and 17.7±0.2 degrees. In one embodiment, the XRPD pattern of esylate salt Form 1 has representative diffraction peaks at diffraction angles 2-theta corresponding to 5.8, 8.7, 16.3, 17.7 and 20.1±0.2 degrees. In one embodiment, the XRPD pattern of esylate salt Form 1 has characteristic diffraction peaks at diffraction angles 2-theta corresponding to 5.8, 8.7, 16.3, 17.7, 20.1, 21.5, 22.3, 23.5, 25.1 and 29.1±0.2 degrees.

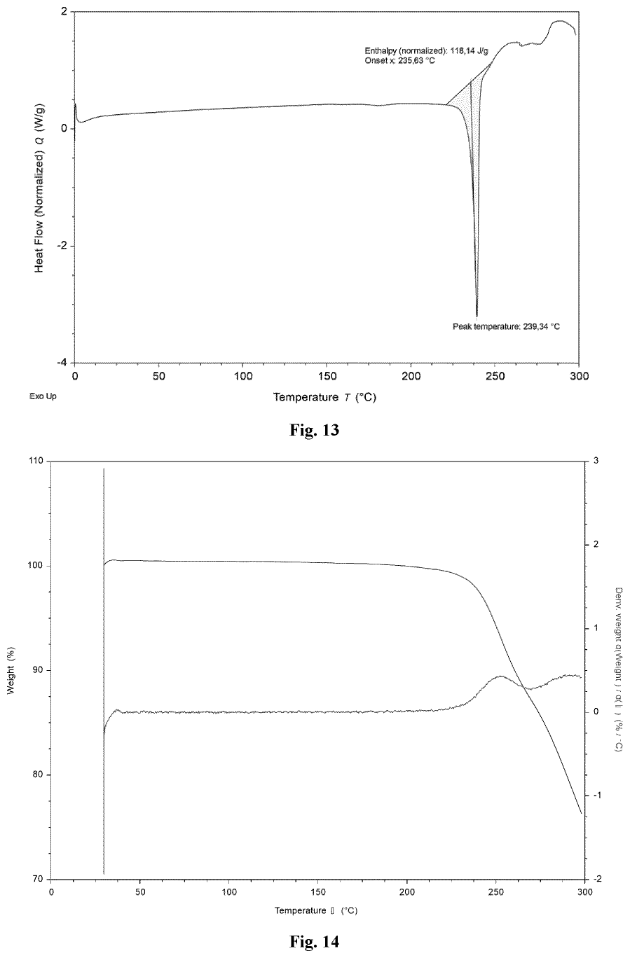

Esylate salt Form 1 exhibits a DSC profile substantially as shown in FIG. 13. Preferably the esylate Form 1 has a differential scanning calorimetry (DSC) onset melting temperature in the range 220-240 °C, more preferably about 235.73 °C.

Esylate salt Form 1 exhibits a TGA profile substantially as shown in FIG. 14. Negligible weight loss was observed until about 237.5 °C, indicating that esylate salt Form 1 is anhydrous.

Esylate salt Form 1 of compound A may be prepared for instance as described in Example 6.

The free base Form A of compound A was used as the starting material for making other forms as described in more detail in the Experimental Part.

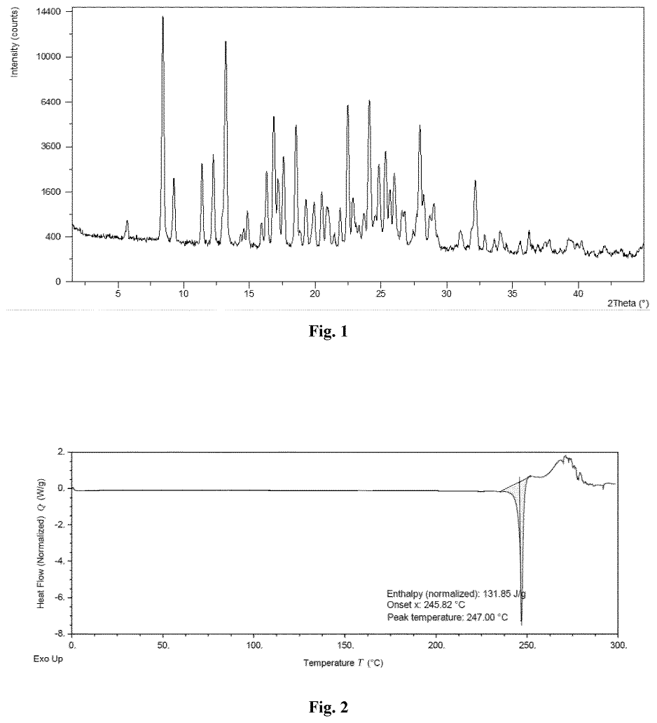

The XRPD pattern of the free base Form A of compound A is substantially as shown in FIG. 1. The XRPD pattern of free base Form A has representative diffraction peaks at diffraction angles 2-theta corresponding to 8.4, 13.1 and 24.1±0.2 degrees. In another embodiment, the XRPD pattern of Form A has representative diffraction peaks at diffraction angles 2-theta corresponding to 8.4, 13.1, 16.8, 22.4, and 24.1±0.2 degrees. In a further embodiment, the XRPD pattern of Form A has characteristic diffraction peaks at diffraction angles 2-theta corresponding to 8.4, 11.3, 12.2, 13.1, 16.8, 18.5, 22.4, 24.1, 25.3 and 27.9±0.2 degrees.

The free base Form A was analysed by proton NMR, as detailed in Example 1. Free base form A has a DSC profile substantially as shown in FIG. 2. The free base Form A is characterized by a DSC profile having a peak at about 247.00 °C.

The free base form A exhibits a TGA profile substantially as shown in FIG. 4. Negligible weight loss was observed until about 250 °C, indicating that free base Form A is anhydrous.

Form A has a DVS profile substantially as shown in FIG. 4. No hygroscopicity was noted as a total of 0.249% water uptake was registered between 20% and 80% RH. The adsorption and desorption profiles are similar, although with some hysteresis between 40% and 90% RH.

In a further aspect, the invention provides a process for the preparation of a salt of Compound A selected from mesylate salt and esylate salt, which comprises reacting the free base of Compound A with methanesulfonic or ethanesulfonic acid.

In one embodiment, the process comprises suspending the free base of Compound A in a polar solvent or polar solvent mixture and adding methanesulfonic or ethanesulfonic acid, either alone or in solution, to cause formation of a crystalline precipitate of the mesylate or esylate salt.

The polar solvent or polar solvent mixture is preferably selected from the group consisting of acetone, tetrahydrofuran, isopropanol, water and mixtures thereof.

The method may further comprise a step of isolating the obtained crystalline precipitate of the mesylate or esylate salt. Preferably, it comprises a step of isolating the crystalline compound A mesylate or esylate salt by filtration.

The method may further comprise a step of washing the filtered crystalline mesylate or esylate salt with one or more polar solvents or solvent mixture, preferably selected from the group consisting of acetone, tetrahydrofuran, isopropanol, water and mixtures thereof.

The method may further comprise a step of recrystallizing the crystalline mesylate or esylate salt with a polar solvent or solvent mixture, preferably selected from the group consisting of acetone, tetrahydrofuran, isopropanol, water and mixtures thereof.

Accordingly, the invention provides a process for the preparation of a crystalline salt of (R)-6-(5-fluoropyridin-2-yl)-8-methoxy-N-(1-(5-methyl-1,2,4-oxadiazol-3-yl)ethyl)quinazolin-4-amine (compound A) selected from mesylate and esylate salt, comprising the steps of:
1) suspending compound A free base in a polar solvent or solvent mixture;
2) adding methanesulfonic or ethanesulfonic acid; and
3) isolating the resulting crystalline salt by filtration.

More preferably, in step 1) the compound A free base is suspended in a polar solvent or solvent mixture selected from the group consisting of acetone, tetrahydrofuran, isopropanol, water and mixtures thereof.

In a preferred embodiment, the process for the preparation of the mesylate salt of Compound A comprises the steps of:
1) suspending compound A free base in a polar solvent or solvent mixture;
2) adding methanesulfonic acid;
   2a) drying the solution obtained in step 2);
   2b) suspending the solid obtained in step 2a) in a polar solvent or solvent mixture and adding an antisolvent thus obtaining the precipitation of a crystalline salt; and
3) isolating the resulting crystalline compound A mesylate salt by filtration.

Preferably, in step 1) and step 2b) the polar solvent or solvent mixture is selected from the group consisting of acetone, tetrahydrofuran, isopropanol, water and mixtures thereof. Also preferably, in step 2b) the antisolvent is heptane.

In another embodiment, the process further comprises a step of washing the filtered crystalline mesylate salt of compound A obtained according to steps 1) to 3) with one or more polar solvents or solvent mixture, preferably selected from the group consisting of acetone, tetrahydrofuran, isopropanol, water and mixtures thereof.

In yet another embodiment, the process further comprises a step of
drying the filtered mesylate salt.

In another preferred embodiment, the process further comprises a step of
recrystallizing the crystalline mesylate salt of compound A from a mixture of isopropanol and water.

In another preferred embodiment, the process for the preparation of the esylate salt of Compound A comprises the steps of:
1) suspending compound A free base in tetrahydrofuran;
2) adding ethanesulfonic acid; and
3) isolating the resulting crystalline compound A esylate salt by filtration.

In yet another preferred embodiment, the process further comprises a step of
washing the filtered crystalline esylate salt of compound A obtained according to steps 1) to 3) with tetrahydrofuran.

In one preferred embodiment, the invention provides a process for the preparation of a salt of (R)-6-(5-fluoropyridin-2-yl)-8-methoxy-N-(1-(5-methyl-1,2,4-oxadiazol-3-yl)ethyl)quinazolin-4-amine (compound A) selected from crystalline mesylate salt Form 2 and crystalline esylate salt Form 1.

The present invention also provides a pharmaceutical composition comprising a mesylate or esylate salt of Compound A and their crystalline forms as described above in admixture with one or more pharmaceutically acceptable carrier or excipient, either alone or in combination with one or more further active ingredient.

In one aspect, the invention refers to a mesylate or esylate salt of Compound A and their crystalline forms as described above according to the invention for use as a medicament.

In a further aspect, the invention refers to the use of a mesylate or esylate salt of Compound A and their crystalline forms, as described above according to the invention, in the manufacture of a medicament for the treatment of disorders associated with P2X₃ receptors mechanism, preferably for the treatment of respiratory diseases.

Preferably, the invention refers to a mesylate or esylate salt of Compound A and their crystalline forms as described above for use in the prevention and/or treatment of respiratory diseases, preferably cough, sub-acute or chronic cough, treatment-resistant cough, idiopathic chronic cough, post-viral cough, iatrogenic cough, asthma, idiopathic pulmonary fibrosis (IPF), chronic obstructive pulmonary disease (COPD) and cough associated with respiratory diseases such as COPD, asthma and bronchospasm.

More preferably, the invention refers to a mesylate or esylate salt of Compound A and their crystalline forms as described above for use in the prevention and/or treatment of chronic cough and cough associated with respiratory diseases such as COPD, asthma and bronchospasm.

The invention also provides a method for the prevention and/or treatment of disorders associated with P2X₃ receptors mechanisms, said method comprising administering to a patient in need of such treatment a therapeutically effective amount of a compound of the invention.

In particular the invention refers to a method for the prevention and/or treatment, wherein the disorder is cough, sub-acute or chronic cough, treatment-resistant cough, idiopathic chronic cough, post-viral cough, iatrogenic cough, asthma, idiopathic pulmonary fibrosis (IPF), chronic obstructive pulmonary disease (COPD) and cough associated with respiratory diseases such as COPD, asthma and bronchospasm, wherein said method comprises the administration of a proper amount of mesylate or esylate salt of Compound A and their crystalline forms as described above to a patient in the need thereof. In a further preferred embodiment, the disorder is chronic cough.

The methods of treatment of the invention comprise administering a safe and effective amount of a mesylate or esylate salt of Compound A and their crystalline forms as described above to a patient in need thereof. As used herein, “safe and effective amount” in reference to a mesylate or esylate salt of Compound A and their crystalline forms as described above or other pharmaceutically-active agent means an amount of the compound sufficient to treat the patient's condition but low enough to avoid serious side effects, such amount being nevertheless routinely determined by the skilled artisan. The mesylate or esylate salt of Compound A and their crystalline forms as described above may be administered once or according to a dosing regimen wherein a number of doses are administered at varying intervals of time for a given period of time. Typical daily dosages may vary depending upon the particular route of administration chosen.

The invention also provides pharmaceutical compositions of mesylate or esylate salt of Compound A and their crystalline forms as described above in a mixture with one or more pharmaceutically acceptable carrier or excipient, for example those described in Remington's Pharmaceutical Sciences Handbook, XVII Ed., Mack Pub., N.Y., U.S.A.

Administration of the mesylate or esylate salt of Compound A and their crystalline forms may be accomplished according to patient needs, for example, orally, nasally, parenterally (subcutaneously, intravenously, intramuscularly, intrasternally and by infusion) and by inhalation.

Preferably, the mesylate or esylate salt of Compound A and their crystalline forms may be administered orally or by inhalation.

Various solid oral dosage forms can be used for administering compounds of the invention including such solid forms as tablets, gelcaps, capsules, caplets, granules, lozenges and bulk powders. The mesylate or esylate salt of Compound A and their crystalline forms can be administered alone or combined with various pharmaceutically acceptable carriers, diluents (such as sucrose, mannitol, lactose, starches) and known excipients, including suspending agents, solubilizers, buffering agents, binders, disintegrants, preservatives, colorants, flavorants, lubricants and the like. Time release capsules, tablets and gels are also advantageous in administering the compounds of the invention.

Preferably, the mesylate or esylate salt of Compound A and their crystalline forms are administered orally. More preferably, the mesylate or esylate salt of Compound A and their crystalline forms are administered in the form of tablets.

Even more preferably, the mesylate salt Form 2 of Compound A is administered in the form of tablets.

Various liquid oral dosage forms can also be used for administering compounds of the invention, including aqueous and non-aqueous solutions, emulsions, suspensions, syrups, and elixirs. Such dosage forms can also contain suitable known inert diluents such as water and suitable known excipients such as preservatives, wetting agents, sweeteners, flavorants, as well as agents for emulsifying and/or suspending the compounds of the invention. The mesylate or esylate salt of Compound A and their crystalline forms may be injected, for example, intravenously, in the form of an isotonic sterile solution.

For the treatment of the diseases of the respiratory tract, the mesylate or esylate salt of Compound A and their crystalline forms may be administered by inhalation.

Inhalable preparations include inhalable powders, propellant-containing metering aerosols or propellant-free inhalable formulations.

For administration as a dry powder, single- or multi-dose inhalers known from the prior art may be utilized. In that case the powder may be filled in gelatine, plastic or other capsules, cartridges or blister packs or in a reservoir.

A diluent or carrier chemically inert to the compounds of the invention, e.g. lactose or any other additive suitable for improving the respirable fraction, may be added to the powdered compounds of the invention.

Inhalation aerosols containing propellant gas such as hydrofluoroalkanes may contain the mesylate or esylate salt of Compound A and their crystalline forms either in solution or in dispersed form. The propellant-driven formulations may also contain other ingredients such as co-solvents, stabilizers and optionally other excipients.

The propellant-free inhalable formulations comprising mesylate or esylate salt of Compound A and their crystalline forms may be in the form of solutions or suspensions in an aqueous, alcoholic or hydroalcoholic medium and they may be delivered by jet or ultrasonic nebulizers known from the prior art or by soft-mist nebulizers.

The mesylate or esylate salt of Compound A and their crystalline forms can be administered as the sole active agent or in combination with other pharmaceutical active ingredients.

Preferably, mesylate or esylate salt of Compound A and their crystalline forms can be combined with therapeutic agents or active ingredients useful for the treatment of disease which are related to or mediated by P2X₃ receptor.

The dosages of mesylate or esylate salt of Compound A and their crystalline forms depend upon a variety of factors including, among others, the particular disease to be treated, the severity of the symptoms, the route of administration, and the like.

The invention is also directed to a device comprising a pharmaceutical composition comprising a mesylate or esylate salt of Compound A and their crystalline forms as described above according to the invention, in form of a single- or multi-dose dry powder inhaler or a metered dose inhaler.

The following examples illustrate the invention without limiting its scope.

### Experimental Part

### Instruments and Methods

### X-Ray Powder Diffraction (XRPD)

The XRPD spectra were collected by X-ray powder diffraction (Empyrean V2.0, Panalytical) equipped with Cu radiation source (Cu Kα 1.5406 Å). Samples were placed on Si zero background sample holders spinning with revolution time 4 s. The measurements were performed in reflection mode, 2-theta scan from 1.5 to 45°, step size 0.02°, soller slit 0.02 rad, divergence slit ⅛°, antiscatter slit ¼°.

The variable temperature and humidity XRPD analysis were carried out with Anton Paar CHC+ camera equipped with CCU100 temperature control and MHG-32 humidity generator. The measurements were performed in reflection mode, 2-theta scan from 1.5 to 45°, step size 0.02°, soller slit 0.02 rad, divergence slit ⅛°, antiscatter slit ¼°.

### Thermogravimetric Analysis (TGA)

TGA analysis was performed using a TA Instruments thermogravimetric analyzer Discovery equipped with a computer analyzing system (TRIOS). Each sample (5-10 mg) was placed in an aluminium sample pan, inserted into the TGA furnace, and accurately weighed. The furnace was first equilibrated at 25 °C, and then heated under nitrogen (flow rate 30 mL/min) at a rate of 10 °C/min, up to a final temperature of 300 °C. Nickel was used as the calibration standard.

### Differential Scanning Calorimetry (DSC)

DSC analysis was performed using a TA Instruments differential scanning calorimeter Discovery equipped with a computer analyzing system (TRIOS).

About 1-5 mg of each sample were placed into a Perkin Elmer Aluminum DSC pan. The pan was covered with a lid, without crimping it. The sample cell was equilibrated at 0 °C and heated under a nitrogen purge (50 mL/min). All samples were given similar thermal histories by linearly heating to 300 °C at a heating rate of 10 °C/min. Indium metal was used as the calibration standard.

### Dynamic Vapour Sorption (DVS)

Moisture sorption/desorption data were collected on a TA Instruments Vapor Sorption Analyzer Q5000SA.

First step: sorption data was collected in the range 40% to 90% relative humidity (RH).

Second Step: desorption, sorption and desorption data were collected over a range of 0% to 90% RH at 10% RH intervals under a nitrogen purge. Samples were not dried prior to analysis. Equilibrium criteria used for analysis were less than 0.100% weight change in 20 minutes, with a maximum equilibration time of 1 hour if the weight criterion was not met. Data were not corrected for the initial moisture content of the samples. NaBr was used as humidity verification.

Moisture sorption/desorption data were also collected on a ProUmid SnS 23 equipped with a 23 autosampler following Method 1.

### Method 1

In the first step sorption data was collected in the range 0% to 90% RH. Samples were not dried prior to analysis.

In the second Step desorption, sorption and desorption data were collected over a range of 0% to 90% (RH) at 10% RH intervals under a nitrogen purge. Equilibrium criteria and cycle parameters are reported in table 1 and table 2. Data were not corrected for the initial moisture content of the samples.

TABLE 1
Measurement conditions

| | | |
|---|---|---|
| Time between weighing cycles | 10.0 | min |
| Min. time per climate cycle | 50.0 | min |
| Max. time per climate cycle | 50.0 | h |

| | |
|---|---|
| Default weight limit | 50.0% |
| Equilibrium condition | 0.0100% per 1.05 min |

TABLE 2
Measurement cycles

| | Humidity | Humidity | | | |
|---|---|---|---|---|---|
| | conditions | conditions | | | |
| Cycle | in % RH | in % RH | No. of | Time in | Temp |
| No. | start value | end value | steps | hours | in °C |
| 1 | 0.0 | 0.0 | 1 | 1.0 | 25.0 |
| 2 | 10.0 | 90.0 | 8 | 1.0 | 25.0 |
| 3 | 80.0 | 0.0 | 8 | 1.0 | 25.0 |
| 4 | 10.0 | 90.0 | 8 | 1.0 | 25.0 |
| 5 | 80.0 | 0.0 | 8 | 1.0 | 25.0 |

### Nuclear Magnetic Resonance Spectroscopy (¹H NMR)

All the ¹H NMR spectra were performed on a Bruker AVANCE III HD 600 spectrometer operating at 600 MHz (proton frequency). The spectrometer was equipped with a 5 mm TCI INVERSE TRIPLE RESONANCE CRYOPROBE H-C/N-D-0.5-Z ATMA. The probe is fitted with an actively shielded single axis Z-gradient and allowed simultaneous decoupling on multiple X-nuclei such as ¹³C and ¹⁵N as well as automatic tuning and matching. Characteristic chemical 10 shifts are reported as 6 values in ppm (parts-per-million). Coupling constants (J values) are given in hertz (Hz) and multiplicities are reported using the following conventional abbreviations for designation of major peaks: s=singlet, d=doublet, t=triplet, q=quartet, dd=doublet of doublets, dt=doublet of triplets, m=multiplet, br=broad, nd=not determined.

UltraPerformance Liquid Chromatography Coupled with UV Detection and Mass Spectrometry (UPLC/UV/MS)

The samples were dissolved in solvent (water/ACN 40/60) at about 1 mg/mL and diluted at about 0.07 mg/mL.

TABLE 3
LC condition

| UPLC instrument: | Waters Acquity |
|---|---|
| Column: | Kinetex C8 100 × 2.1 mm, 1.7 μm (Phenomenex) |
| Column | 55 |
| Temperature (°C): | |
| Mobile phases: | CH₃COONH₄ 0.01M pH 5 (A); MeCN + 0.1% |
| | HCOOH (B) |
| Flow (mL/min): | 0.5 |
| Stop Time (mins): | 10 |

Gradient:

| Time (min) | % A | % B |
|---|---|---|
| 0.00 | 99.0 | 1.0 |
| 0.50 | 99.0 | 1.0 |
| 3.00 | 70.0 | 30.0 |
| 6.50 | 50.0 | 50.0 |
| 7.50 | 20.0 | 80.0 |
| 8.00 | 20.0 | 80.0 |
| 8.10 | 99.0 | 1.0 |
| 10.00 | 99.0 | 1.0 |

TABLE 4
UV condition

| | |
|---|---|
| UV detection: | DAD |
| UV acquisition range (nm): | 210-400 |
| Injection Volume (μL): | 2 |
| Sample Solvent: | Acetonitrile/Water 60/40 |

TABLE 5
MS condition

MS instrument: Waters QDA
Acquisition Mode: Fullscan MS scan
Polarity: ES+/ES−
Mass range: 30-1250

### Crystallization Systems

Polar Bear Plus, Cambridge Reactor Design was used for slurry at 50 °C and temperature cycling experiments while for crystallization optimization Crystal16 (Technobis Crystallization systems) was used.

### Example 1: Characterisation of the Free Base Form A of Compound A

The method of synthesis of free base Form A of compound A is described in WO2020239951 A1 (Example 315, page 231-2).

The free base Form A of compound A was characterized by XRPD, proton NMR, DSC, TGA and DVS.

The free base Form A is consistent with an anhydrous form of compound A. A proton NMR spectrum for the material is consistent with the chemical structure of Compound A free base. The characteristic chemical shifts are reported herebelow:

¹H NMR (600 MHz, DMSO-d6) δ ppm 1.68 (d, J=6.92 Hz, 3H) 2.56 (s, 3H) 4.00 (s, 3H) 5.76 (quin, J=7.24 Hz, 1H) 7.94-8.00 (m, 2H) 8.29 (dd, J=8.98, 4.36 Hz, 1H) 8.46 (s, 1H) 8.57 (d, J=1.80 Hz, 1H) 8.70-8.72 (m, 1H) 8.70-8.75 (m, 1H)

The XRPD pattern of free base Form A of compound A is shown in FIG. 1 and the complete peak list is shown here below.

Form A - XRPD [degrees 2-theta]

5.7
8.4
9.2
11.3
12.2
13.1
14.3
14.6
14.9
15.9
16.3
16.8
17.2
17.6
18.5
19.3
19.9
20.5
20.9
21.1
21.4
21.9
22.4
22.9
23.7
24.1
24.8
25.3
25.7
26.0
26.6
26.8
27.9
28.2
28.7
29.0
31.0
32.2
32.9
33.6
34.1
35.6
36.3
37.8
39.3
40.2
41.1
42.0
43.3

The following characteristic diffraction peaks at diffraction angles 2-theta were identified for free base Form A of compound A: 8.4, 11.3, 12.2, 13.1, 16.8, 18.5, 22.4, 24.1, 25.3 and 27.9±0.2 degrees. A selection of five representative diffraction peaks at diffraction angles 2-theta were identified from the characteristic diffraction peaks: 8.4, 13.1, 16.8, 22.4 and 24.1±0.2 degrees. A further selection of three representative diffraction peaks at diffraction angles 2-theta were identified from the characteristic diffraction peaks: 8.4, 13.1 and 24.1±0.2 degrees.

A DSC profile of free base Form A is shown in FIG. 2. The DSC profile of free base Form A displays one enthalpy event having an onset at 245.82 °C and a peak at 247.00 °C. The peak at 247.00 °C indicates the melting temperature of the free base Form A.

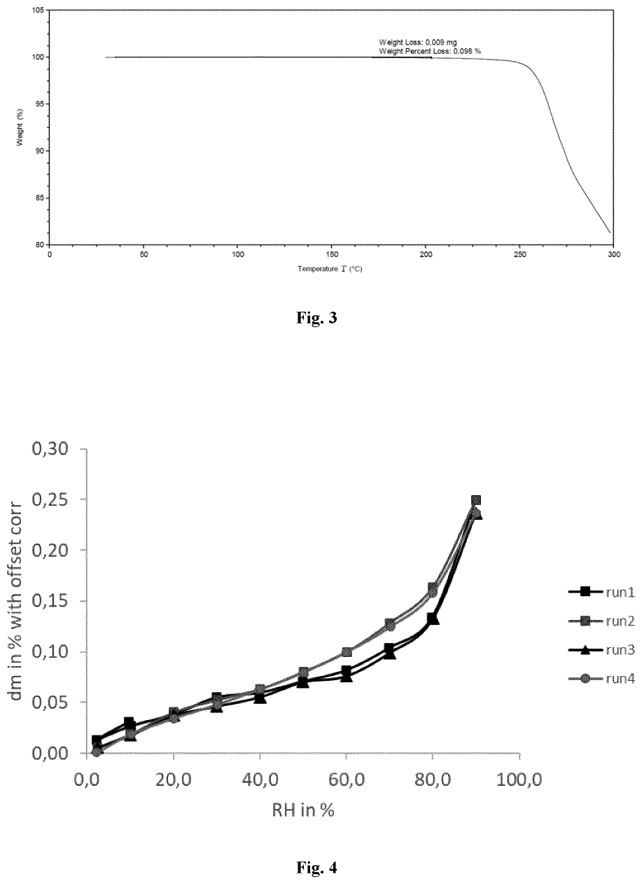

A TGA profile of free base Form A is shown in FIG. 3. Negligible weight loss was observed until around 250 °C, corresponding to the melting temperature observed in the DSC profile. This weight loss continued until the end of measurements at 300 °C. The TGA profile is indicative of the anhydrous nature of free base Form A.

A DVS profile of free base Form A is shown in FIG. 4. Hygroscopicity was noted between 2.5% and 90% RH, with the sample gaining a total of about 0.249 wt %. The adsorption and desorption profiles are similar, although with some hysteresis between 40% and 90% RH.

### Example 2: Preparation and Characterization of the Mesylate Salt Form 2 of Compound A

### Preparation of the Mesylate Salt Form 2 of Compound A—Method A

In a round-bottomed flask, 150 mg of compound A according to Example 1 (free base Form A, 0.39 mmol) was suspended in acetone (15 mL) and stirred at RT exposed to ambient humidity. Then methanesulfonic acid (25 μL, 0.39 mmol) was added and let stirring in closed vial. After 2 hours the crystalline solid was isolated by filtration, washed with acetone (2×5 ml) and dried at room temperature for 2 hours (95% yield). The acetone in this preparation was commercial and used without further distillation or purification (water amount >0.1%).

### Preparation of the Mesylate Salt Form 2 of Compound A—Method B

In a round-bottomed flask, 150 mg of compound A according to Example 1 (free base Form A, 0.39 mmol) was suspended in tetrahydrofuran (THF) (15 mL) and stirred at RT exposed to ambient humidity. Then methanesulfonic acid (25 μL, 0.39 mmol) was added while stirring and the solution was dried under nitrogen at RT. The solid obtained was suspended in acetone and heptane was added as antisolvent. The crystalline solid was isolated by filtration and dried at room temperature (90% yield).

The solvents in this preparation were commercial and used without further distillation or purification (water amount >0.1%).

### Preparation of the Mesylate Salt Form 2 of Compound A—Method C

In a round-bottomed flask, 5 g of compound A according to Example 1 (free base Form A) were suspended in isopropanol (47.5 mL). The mixture was stirred and heated up to reflux, then a mixture of methanesulfonic acid (1.5 mL) and water (2.5 mL) was added dropwise. The solution was cooled down to 0 °C in 3 h and left under stirring at this temperature for 6 h. The solid was filtered over a gooch filter and washed with isopropanol (15 mL). Yield 78%.

### Preparation of the Mesylate Salt Form 2 of Compound A—Method D

In a round-bottomed flask, 7.5 g of the mesylate salt of compound A, prepared according to Method A, B or C, were suspended in a solvent mixture of isopropanol (71.25 mL) and water (3.75 mL). The mixture was stirred and heated up to reflux, then cooled down to 55 °C, the slurry aged at this temperature for 30 min then cooled down to 10 °C in 3 h and left under stirring at this temperature for 6 h. The solid was filtered over a gooch filter and washed with isopropanol (15 mL). Yield 80%.

### Characterization of the Mesylate Salt Form 2 of Compound A

The XRPD pattern of the mesylate salt Form 2 of compound A obtained according to Method A or Method B is shown in FIG. 5 and the complete peak list is shown herebelow:

Form 2 - XRPD [degrees 2-theta]

6.6
9.3
11.8
13.3
14.9
15.6
17.2
17.7
18.1
18.5
19.0
19.2
20.0
21.1
22.4
23.5
23.8
24.4
25.7
26.8
27.4
28.0
28.5
30.1
31.1
31.5
31.8
33.4
34.6
35.7
36.5
37.5
39.2
40.6
41.6
43.5
43.9
44.3

The mesylate salt Form 2 was analysed by proton NMR. The spectrum was consistent with a 1:1 mesylate salt Form 2 of compound A. The characteristic chemical shifts are reported herebelow:

¹H NMR (600 MHz, DMSO-d₆) δ ppm 1.76 (d, J=7.16 Hz, 3H), 2.30 (s, 3H), 2.60 (s, 3H), 4.17 (s, 3H), 5.96 (quin, J=7.16 Hz, 1H), 8.04 (dt, J=8.72, 3.11 Hz, 1H), 8.25 (d, J=1.25 Hz, 1H), 8.32 (dd, J=9.03, 4.36 Hz, 1H), 8.78 (d, J=2.80 Hz, 1H), 8.81 (d, J=1.25 Hz, 1H), 8.83 (s, 1H), 10.52 (br s, 1H), 13.75-15.33 (br s, 1H).

A DSC profile of mesylate salt Form 2 is shown in FIG. 6. The first enthalpy event displayed an onset at 22.95 °C with a peak at 73.86 °C. The second enthalpy event displayed an onset at 232.13 °C with a peak at 237.25 °C. The peak at 237.25 °C indicates the melting temperature of the mesylate salt Form 2.

A TGA profile of mesylate salt Form 2 is shown in FIG. 7. An initial weight loss step of 1.487% occurred between about 37.5 °C and 75 °C. Little to no weight loss was observed between 75 °C and 237.5 °C, followed by a substantial weight loss corresponding to the melting temperature observed in the DSC profile. The weight loss continued until the end of measurements at 300 °C.

A DVS profile of mesylate salt Form 2 is shown in FIG. 8. Hygroscopicity was noted between 0% and 90% RH, with the sample gaining a total of about 1.049 wt %. Approximately half of the weight gain occurred between 0% and 15% RH. The adsorption and desorption profiles are similar, although with some hysteresis between 40% and 80% RH. Approximately half of the weight loss occurred between 0% and 15% RH.

### Example 3: Preparation and Characterisation of the Mesylate Salt Form 3 of Compound A

The anhydrous mesylate Form 3 was obtained from the hemihydrate mesylate Form 2 by dehydration. Dehydration began at 75 °C and the Form 3 appeared.

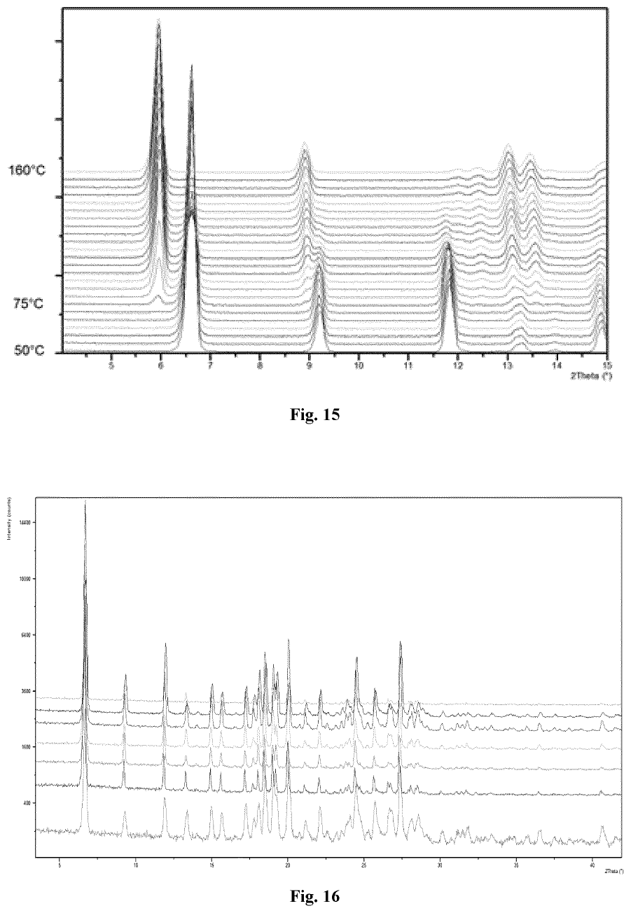

The complete dehydration seemed to be achieved at 160 °C, as shown in a variable temperature XRPD (VT-XRPD) pattern of mesylate salt Form 2 transitioning to mesylate salt Form 3 of compound A from 50 °C to 160 °C collected every 5 °C from 50 °C in FIG. 15.

The XRPD pattern of mesylate salt Form 3 of compound A is shown in FIG. 9. The DSC profile of mesylate salt Form 3 displayed one enthalpy event having an onset at 229.65 °C and a peak at 232.02 °C. The peak at 232.03 °C indicates the melting temperature of the mesylate salt Form 3.

The TGA profile of mesylate salt Form 3 shows negligible weight loss until around 237.5 °C, corresponding to the melting temperature observed in the DSC profile. This weight loss continued until the end of measurements at 300 °C. The TGA profile is indicative of the anhydrous nature of Form 3.

### Example 4: Preparation and Characterisation of the Mesylate Salt Form 4 of Compound A

An anhydrous mesylate salt Form 4 was scaled up by slurrying 10 mg/ml of mesylate salt Form 2 in acetone for at least 24 h, filtered, rinsed with diethyl ether and dried under nitrogen. Calorimetric analyses on Form 4 showed an endo peak at roughly 145 °C (data not shown).

The XRPD pattern of mesylate salt Form 4 of compound A is shown in FIG. 10 and the complete peak list is shown herebelow:

Form 4 - XRPD [degrees 2-theta]

6.0
8.8
13.6
15.6
16.1
16.5
17.0
17.7
18.9
20.1
21.1
21.7
22.0
23.2
24.1
24.7
26.7
27.5
27.8
28.2
28.9
29.2
29.4
29.7
31.3
32.3
33.0
34.3
34.9
35.1
35.5
35.8
37.3
40.6
41.3
42.6
43.1
43.5

The DSC profile of mesylate salt Form 4 displayed two enthalpy events. The first enthalpy event is a minor event having an onset at 141.47 °C and a peak at 141.97 °C. The second enthalpy event has an onset at 236.74 °C and a peak at 238.93 °C. The peak at 238.93 °C indicates the melting temperature of the mesylate salt Form 4.

The TGA profile of mesylate salt Form 4 shows negligible weight loss until around 240 °C, corresponding to the melting temperature observed in the DSC profile. This weight loss continued until the end of measurements at 300 °C. The TGA profile is indicative of the anhydrous nature of mesylate salt Form 4.

### Example 5: Preparation and Characterisation of the Mesylate Salt Form 6 of Compound A

### Preparation of the Mesylate Salt Form 6 of Compound A—Method E

Mesylate salt Form 2 was stirred in methyl ethyl ketone (MEK) at 40 °C overnight (10 mg/ml), filtered, rinsed with diethyl ether and dried under nitrogen flux.

### Characterisation of the Mesylate Salt Form 6 of Compound A

The XRPD pattern of mesylate salt Form 6 of compound A obtained according to Method E is shown in FIG. 11 and the complete peak list is shown here below:

Form 6 - XRPD [degrees 2-theta]

3.4
6.8
8.5
8.9
11.8
12.6
13.7
14.1
16.3
16.6
16.9
17.2
18.0
18.3
18.9
19.2
20.0
20.6
21.0
21.7
22.1
22.4
22.9
23.7
24.3
24.7
25.2
25.4
26.0
26.7
27.1
27.6
28.4
29.0
31.3
35.7
36.1
38.6

The DSC profile of mesylate salt Form 6 displayed two enthalpy events. The first enthalpy event has an onset at 221.51 °C and a peak at 226.50 °C. The second enthalpy event has an onset at 235.43 °C and a peak at 237.96 °C. The peak at 237.96 °C indicates the melting temperature of the mesylate salt Form 6.

The TGA profile of mesylate salt Form 6 has shown negligible weight loss of about 0.2% until around 237.5 °C, corresponding to the melting temperature observed in the DSC profile. This weight loss continued until the end of measurements at 300 °C. The TGA profile is indicative of the anhydrous nature of mesylate salt Form 6.

The DVS profile of mesylate salt Form 6 has shown substantially no hygroscopicity as a total of 0.3% water uptake was registered between 20% and 80% RH.

### Example 6: Preparation and Characterisation of the Esylate Salt Form 1 of Compound A

In a round-bottomed flask, 150 mg of compound A free base according to Example 1 (0.39 mmol) was suspended in THF (5 mL) and stirred at RT. Then an excess of ethanesulfonic acid (32 μL, 0.395 mmol) was added while stirring and let concentrate in open vial. After 1 hour the crystalline solid was isolated by filtration, washed with THE (2×1 ml) and dried under nitrogen for 2 hours (80% yield).

The XRPD pattern of the esylate salt Form 1 of compound A is shown in FIG. 12 and the complete peak list is shown herebelow:

Form 1 - XRPD [degrees 2-theta]

5.8
8.7
13.7
16.0
16.3
16.9
17.7
19.0
20.1
20.9
21.5
22.3
23.5
24.0
25.1
26.8
27.4
27.8
28.2
28.8
29.1
32.3
32.9
34.6
35.5
37.1
38.7
40.6

The esylate salt Form 1 was analysed by proton NMR. The spectrum was consistent with a 1:1 esylate salt Form 1 of compound A. The characteristic chemical shifts are reported herebelow:

¹H NMR (400 MHz, DMSO-d₆) δ ppm 1.05 (3H, t, J=7.50 Hz), 1.75 (3H, d, J=7.06 Hz), 2.36 (2H, q, J=7.35 Hz), 2.60 (3H, s), 4.17 (3H, s), 5.96 (1H, t, J=7.28 Hz), 8.04 (1H, td, J=8.71, 3.09 Hz), 8.24 (1H, s), 8.32 (1H, dd, J=8.82, 4.41 Hz), 8.80 (3H, m), 10.48 (1H, br s).

A DSC profile of esylate salt Form 1 is shown in FIG. 13. The DSC profile of esylate salt Form 1 displayed one enthalpy event having an onset at 235.735 °C and a peak at 239.343 °C. The peak at 239.343 °C indicates the melting temperature of the esylate salt Form 1.

A TGA profile of esylate salt Form 1 is shown in FIG. 14. Negligible weight loss was observed until about 237.5 °C, corresponding to the melting temperature observed in the DSC profile. This weight loss continued until the end of measurements at 300 °C. The TGA profile is indicative of the anhydrous nature of the esylate salt Form 1 of compound A.

### Example 7: Stability of the Mesylate Salt Form 2 of Compound A

Approximately 120 mg of mesylate salt Form 2 of Compound A according to Example 2 was stored in a properly labelled open amber glass vial covered with filter paper to simulate open conditions, and in a screw cap glass to simulate closed conditions. The samples were prepared such that a layer of powder was uniformly spread on the bottom of the vial. Approximately 70 mg of powder were weighed in hydroxypropyl methylcellulose (HPMC) capsules size 3 (Capsugel) and for each condition two capsules were stored. The samples were placed at RT in a desiccator with NaBr to reach about 60% RH and in a stability chamber to reach 75% RH at 40 °C. The stability of the mesylate salt Form 2 of Compound A was thereby tested in four distinct conditions: open condition at 25 °C/60% RH, closed condition at 25 °C/60% RH, open condition at 40 °C/75% RH, and closed condition at 40 °C/75% RH. At the time points 0, 14, 28, 56, 84, 168 and 365 days the corresponding samples were removed from the stability chambers. After conditioning at RT, the samples were analyzed by UPLC/UV/MS and XRPD. One or two extra samples were stored for every condition.

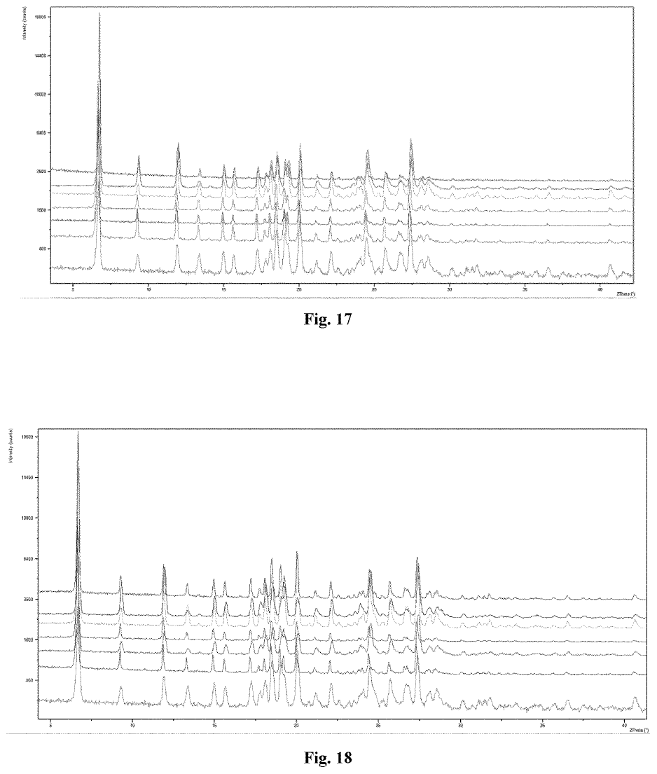

The results from the 25 °C/60% RH open and closed conditions are shown in Table 6, FIG. 16 and FIG. 17. The XRPD patterns shown in FIG. 16 and FIG. 17 show no significant changes and demonstrate that mesylate salt Form 2 of Compound A remained stable throughout the entire test period in both the open condition, FIG. 16, and the closed condition, FIG. 17. The mesylate salt Form 2 thereby demonstrated excellent stability for at least one year at RT.

TABLE 6
Stability at 25 °C and 60% RH at open and closed conditions

| | 25 °C/60% RH Open | | | 25 °C/60% RH Closed | | |
|---|---|---|---|---|---|---|
| | LC/UV/MS | | | LC/UV/MS | | |
| Time | (% area main peak) | | | (% area main peak) | | |
| (days) | UV | ES+ | ES− | UV | ES+ | ES− |
| 0 | 98.4 | 91.6 | 100.0 | 98.4 | 91.6 | 100.0 |
| 14 | 98.4 | 90.6 | 100.0 | 98.5 | 90.1 | 100.0 |
| 28 | 98.3 | 89.6 | 100.0 | 98.3 | 89.9 | 100.0 |
| 56 | 98.4 | 93.0 | 100.0 | 98.4 | 92.8 | 100.0 |
| 84 | 98.4 | 90.1 | 100.0 | 98.4 | 90.3 | 100.0 |
| 168 | 98.3 | 90.6 | 100.0 | 98.3 | 90.4 | 100.0 |
| 365 | 98.3 | 92.1 | 100.0 | 98.3 | 93.1 | 100.0 |

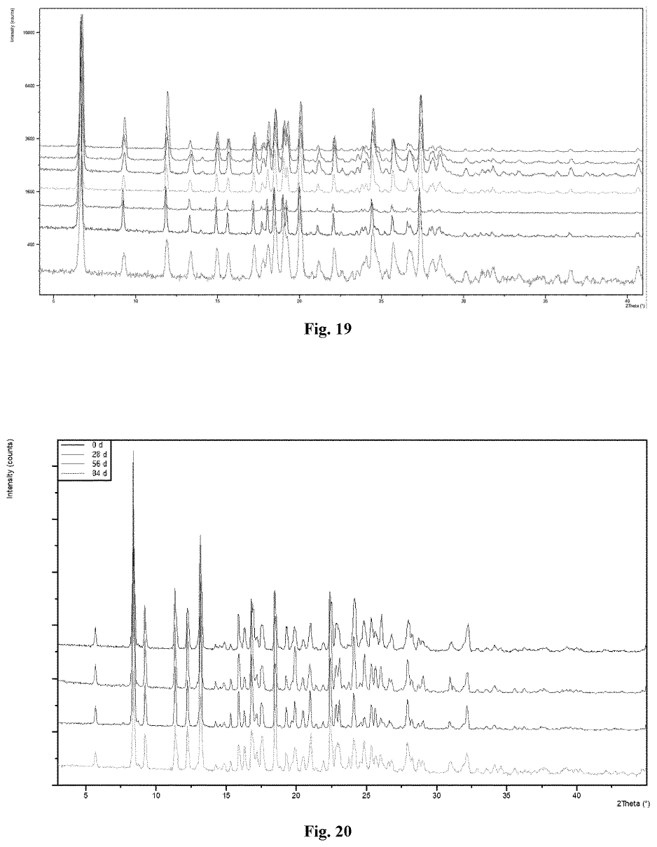

The results from the 40 °C/75% RH open and closed conditions are shown in Table 7, FIG. 18 and FIG. 19. The XRPD patterns shown in FIG. 18 and FIG. 19 show no significant changes and demonstrate that mesylate salt Form 2 of Compound A remained stable throughout the entire test period in both the open condition, FIG. 18, and the closed condition, FIG. 19.

TABLE 7
Stability at 40 °C and 75% RH at open and closed conditions

| | 40 °C/75% RH Open | | | 40 °C/75% RH Closed | | |
|---|---|---|---|---|---|---|
| | LC/UV/MS | | | LC/UV/MS | | |
| Time | (% area main peak) | | | (% area main peak) | | |
| (days) | UV | ES+ | ES− | UV | ES+ | ES− |
| 0 | 98.4 | 91.6 | 100.0 | 98.4 | 91.6 | 100.0 |
| 14 | 98.4 | 90.6 | 100.0 | 98.4 | 90.5 | 100.0 |
| 28 | 98.3 | 85.9 | 100.0 | 98.4 | 89.8 | 100.0 |
| 56 | 98.4 | 92.5 | 100.0 | 98.4 | 90.4 | 100.0 |
| 84 | 98.3 | 90.9 | 100.0 | 98.4 | 91.8 | 100.0 |
| 168 | 98.4 | 91.1 | 100.0 | 98.3 | 91.5 | 100.0 |
| 365 | 98.3 | 92.0 | 100.0 | 98.4 | 92.2 | 100.0 |

The above results demonstrate that the mesylate salt Form 2 of compound A has a surprisingly good stability during conditions associated with regular storage and stressed storage conditions. The mesylate salt Form 2 of compound A remains stable in dry form for at least one year at different conditions.

In a second experiment, approximately 100 mg of compound A mesylate salt Form 2 according to Example 2, free base Form A according to Example 1 and esylate salt Form 1 according to Example 6 were stored in three open amber glass vials covered with filter paper to simulate open conditions. The samples were stored in a stability chamber at 75% RH and 40 °C to compare their stability. At the time points 0, 28, 56, 84 days the corresponding samples were removed from the stability chambers. After conditioning at RT, the samples were analyzed by UPLC/UV and XRPD.

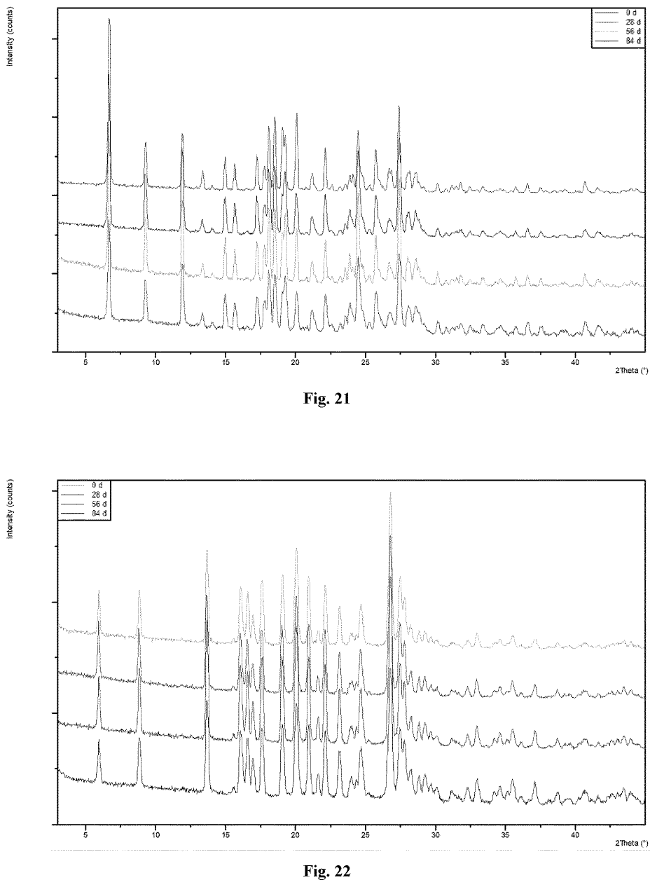

The results are reported in table 7A. The XRPD patterns are shown in FIGS. 20, 21 and 22. Both mesylate salt Form 2 and esylate salt Form 1 of compound A demonstrate a surprisingly good stability during stressed storage.

TABLE 7A
Comparative stability at 40 °C and 75% RH at open conditions

| | Free base | Mesylate salt | Esylate salt |
|---|---|---|---|
| | form A | Form 2 | Form 1 |
| Time | LC/UV | LC/UV | LC/UV |
| (days) | (% area main peak) | (% area main peak) | (% area main peak) |
| 0 | 99.8 | 99.9 | 99.9 |
| 28 | 99.8 | 99.9 | 99.9 |
| 56 | 99.8 | 99.9 | 99.9 |
| 84 | 99.8 | 99.9 | 99.9 |

### Example 8: Dissolution of Mesylate Salt Form 2 and Esylate Salt Form 1 of Compound A in Fasted State Simulated Gastric Fluid (FaSSGF) in Comparison with Free Base Form A and Other Salts

The dissolution in Fasted State Simulated Gastric Fluid (FaSSGF) of mesylate salt Form 2 of the compound A of Example 2 and esylate salt Form 1 of Example 6 in comparison with the free base Form A and with other salts was tested by means of manual experiment. The medium for simulated oral fluids was FaSSGF obtained from Biorelevant.com Ltd. The experiments were conducted for 60 minutes at 37 °C. The FaSSGF medium composition is listed in Table 9.

General preparation of salts: in a round-bottomed flask, 200 mg of compound A free base Form A according to Example 1 (0.52 mmol) were suspended in 5 ml of solvent and stirred at RT. Acetone was employed for bromide, ethanedisulfonate, malonate, sulfate, saccharinate, tosylate, orotate, besylate, hemisulfate, phosphate and tartrate salts; water was employed for chloride salt; ethyl formate was employed for maleate, oxalate, gentisicate, fumarate and mandelate salts; tetrahydrofuran was employed for camphorsulfonate and pamoate salts; isopropyl acetate was employed for 4-hydroxybenzoate and methanol for camphorate salt. The corresponding acid was added under stirring in equimolar amount (except for hemisulfate, added in 0.5 molar amount). After 1 hour the crystalline solid was isolated by filtration, washed with the corresponding solvent (2×1 ml) and dried under nitrogen for 2 hours.

The quantification of the dissolved amount was done by LC/UV system at different checkpoints up to 60 minutes. The saturation solution concentration was around 25-30 mg/ml. Mesylate salt Form 2 and esylate salt Form 1 of Compound A were completely dissolved. The dissolution results are reported in Table 8. In a second experiment, the quantification was repeated with saturation solution concentration 170 mg/ml for the mesylate salt Form 2 and 100 mg/ml for the esylate salt Form 1. The results are reported in tables 8A, 8B, 8C, 8D and FIG. 28.

TABLE 8
Dissolution in FaSSGF

| | Solubility (mg/ml) | | | | | |
|---|---|---|---|---|---|---|
| Time | free base | mesylate | esylate | | | |
| (min) | Form A | Form 2 | Form 1 | oxalate | saccharinate | fumarate |
| 5 | 6.87 | 31.44 | 24.27 | 0.09 | 0.90 | 3.29 |
| 10 | 7.01 | 31.92 | 24.74 | 0.09 | 1.03 | 3.36 |
| 20 | 6.84 | 31.65 | 24.87 | 0.08 | 1.09 | 3.35 |
| 40 | 6.89 | 31.16 | 24.76 | 0.09 | 1.08 | 3.61 |
| 60 | 6.89 | 31.30 | 24.59 | 0.09 | 1.02 | 3.58 |

TABLE 8A
Dissolution in FaSSGF, second experiment

| | Solubility (mg/ml) | | | | | |
|---|---|---|---|---|---|---|
| Time | free base | mesylate | esylate | | | |
| (min) | Form A | Form 2 | Form 1 | oxalate | saccharinate | fumarate |
| 0 | 0.63 | 19.25 | n/a | 1.73 | 0.43 | 0.55 |
| 15 | 12.77 | 151.67 | n/a | 10.71 | 0.80 | 4.98 |
| 30 | 11.89 | 159.96 | 61.30 | 11.70 | 0.87 | 5.76 |
| 60 | 13.47 | 169.82 | 68.46 | 13.87 | 0.83 | 5.89 |

TABLE 8B
Dissolution in FaSSGF, second experiment

| | Solubility (mg/ml) | | | | | |
|---|---|---|---|---|---|---|
| | | | 4- | | | |
| Time | phos- | camphor- | hydroxy- | | | |
| (min) | phate | sulfonate | benzoate | mandelate | tosylate | camphorate |
| 0 | 0.21 | 0.51 | 1.73 | 1.28 | 0.61 | 1.63 |
| 15 | 15.21 | 10.51 | 10.97 | 10.88 | 4.67 | 10.42 |
| 30 | 14.85 | 11.84 | 11.37 | 9.30 | 4.86 | 10.50 |
| 60 | 14.32 | 13.45 | 11.69 | 10.99 | 4.97 | 10.83 |

TABLE 8C
Dissolution in FaSSGF, second experiment

| | Solubility (mg/ml) | | | | | |
|---|---|---|---|---|---|---|
| Time | | | | ethane- | | |
| (min) | pamoate | bromide | chloride | disulfonate | besylate | gentisicate |
| 0 | 0.03 | 0.72 | 0.69 | 0.49 | 1.01 | 1.97 |
| 15 | 4.89 | 2.52 | 8.63 | 1.10 | 21.74 | 11.42 |
| 30 | 6.10 | 2.70 | 9.42 | 1.09 | 21.84 | 13.04 |
| 60 | 6.52 | 2.88 | 10.87 | 1.06 | 21.84 | 14.04 |

TABLE 8D
Dissolution in FaSSGF, second experiment

| Time | Solubility (mg/ml) | | | | | |
|---|---|---|---|---|---|---|
| (min) | malonate | maleate | tartrate | hemisulfate | sulfate | orotate |
| 0 | 2.88 | 0.67 | 0.42 | 1.08 | 2.00 | 1.16 |
| 15 | 3.97 | 2.72 | 5.58 | 4.84 | 3.54 | 3.49 |
| 30 | 4.25 | 3.08 | 6.38 | 4.77 | 3.14 | 4.23 |
| 60 | 4.24 | 2.65 | 6.44 | 4.57 | 3.29 | 4.71 |

As it can be appreciated, the mesylate salt Form 2 and the esylate salt Form 1 of compound A showed a consistently higher solubility than the free base Form A during the 60 minutes time. Their solubility was surprisingly higher than that of the other tested salts, as reported in Table 8, 8A, 8B, 8C and 8D. The solubility of salts other than mesylate and esylate proved to be lower or, at best, comparable to the solubility of free base Form A.

### Example 9: Dissolution of Mesylate Salt Form 2 of the Compound a in all Simulated Oral Fluids

The dissolution of mesylate salt Form 2 of the compound A in comparison with the free base Form A of compound A was tested in all simulated oral fluids. The experiments were conducted with DISS Profiler 6 Channel Pion. The experiments were conducted until 24 hours at 37 °C.

The mediums used, namely FaSSGF (Fasted State Simulated Gastric Fluid), FaSSIF (Fasted State Simulated Intestinal Fluid) and FeSSIF (Fed State Simulated Intestinal Fluid) were obtained from Biorelevant.com Ltd. The medium compositions are listed in table 9. The saturated solution concentrations were about 14-15 mg/ml (as free base) in FaSSGF and about 3-4 mg/ml (as free base) in FaSSIF and FeSSIF.

TABLE 9
Composition of media used for dissolution studies

| Test Fluid | FaSSIF | FeSSIF | FaSSGF |
|---|---|---|---|
| pH | 6.5 | 5.0 | 1.6 |
| Sodium taurocholate (mM) | 3.0 | 15.0 | 0.08 |
| Lecithin (mM) | 0.75 | 3.75 | 0.02 |
| Sodium chloride (mM) | 106 | 203 | 34.2 |
| Monobasic sodium phosphate (mM) | 28.4 | — | — |
| Sodium hydroxide (mM) | 8.7 | 101 | — |
| Acetic acid (mM) | — | 144 | — |
| Hydrochloric acid (mM) | — | — | ≈25.1 |

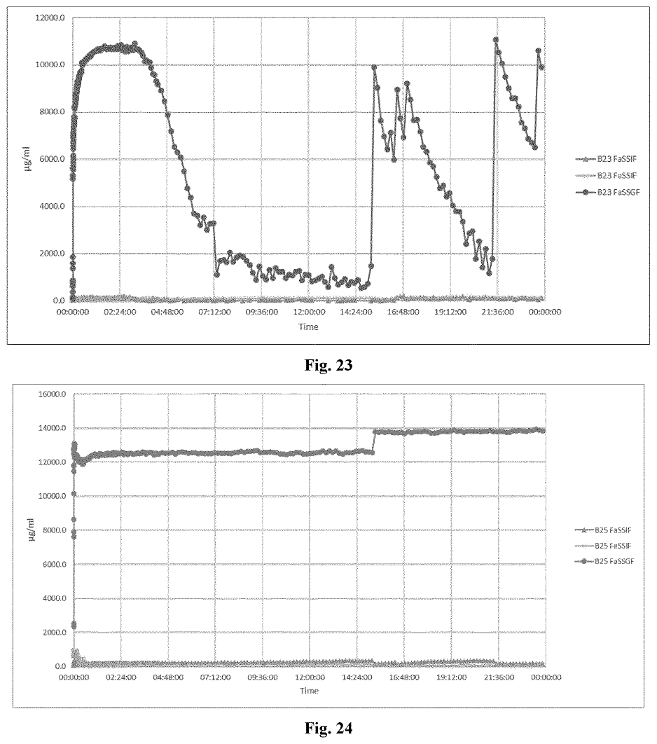

The results for free base Form A are shown in FIG. 23. In FaSSGF, the free base Form A of compound A showed a steady increase of solubility, but not complete solubilisation. The solubility in FaSSGF was about 11 mg/ml. A decrease was subsequently observed after approximately 3 hours.

A lower solubility was observed in FaSSIF and FeSSIF of 0.02 mg/ml and 0.1 mg/ml, respectively.

The maximum solubility values obtained during the experiment and the end pH for the free base Form A of compound A are reported in Table 10.

TABLE 10
Solubility - free base Form A of compound A

| Medium | Maximum solubility (mg/ml) | pH |
|---|---|---|
| FaSSGF | 11.00 | 3.1 |
| FaSSIF | 0.04 | 6.4 |
| FeSSIF | 0.10 | 5.1 |

The results for mesylate salt Form 2 of compound A are shown in FIG. 24. As can be appreciated for mesylate salt Form 2 of compound A, complete solubilisation was achieved in FaSSGF at a maximum solubility of 14.00 mg/ml. There was no decrease in the solubility during the 24 hours.

A lower maximum solubility was observed in FaSSIF and FeSSIF than FaSSGF of 0.10 mg/ml and 0.07 mg/ml, respectively.

The maximum solubility values obtained during the experiment and the pH found at the end of the experiment for the mesylate salt Form 2 of compound A are reported in table 11.

The lower maximum solubility in FaSSIF and FeSSIF was also confirmed by UPLC/UV/MS at the end of the experiment giving analogous results, 0.03 mg/ml and 0.17 mg/ml, respectively.

TABLE 11
Solubility - mesylate salt Form 2 of compound A

| Medium | Maximum solubility (mg/ml) | pH |
|---|---|---|
| FaSSGF | 14.00 | 1.8 |
| FaSSIF | 0.10 | 6.1 |
| FeSSIF | 0.07 | 5.0 |

The above results demonstrate that the mesylate salt Form 2 of compound A leads to an unexpected increase in maximum solubility compared to the free base Form A of compound A in FaSSGF.

Furthermore, the mesylate salt Form 2 of compound A unexpectedly demonstrates a sustained solubility for at least 24 hours compared to the drop in solubility for the free base Form A of compound A after approximately 3 hours.

The solubility of the mesylate salt Form 2 of compound A in FaSSGF is therefore particularly advantageous in gastric fluid, potentially facilitating also the subsequent uptake.

### Example 10: Two-Sector Dissolution Study

The dissolution behaviour of mesylate salt Form 2 of compound A was studied in a two-sector model in comparison with free base Form A to evaluate a possible “spring-parachute effect”.

The parachute effect is observable in two-sector dissolution studies. A test compound is incubated with FaSSGF and its concentration is monitored over time. FaSSIF is then added to the solution whilst continuing the monitoring. The period of time in which the test compound remains in solution without or before precipitating refers to the supersaturation time and represents the parachute effect.

The experiment was conducted using DISS Profiler 6 Channel Pion. The free base Form A and mesylate salt Form 2 of compound A were each incubated in 8 ml of FaSSGF (pH 1.6) for 20 minutes at the concentration of 350 mg/250 ml. 16 ml of FaSSIF concentrated 1.5× (pH 6.5) was added with an incubation time of 180 minutes. The resulting target pH should be approximately 5-6. The experiment was conducted at 37 °C. The entire dissolution profile was monitored until the end of the experiment to evaluate a possible parachute effect, its duration and to evaluate the maximum solubility reached after the addition of FaSSIF.

Free base Form A of compound A was tested in duplicate and the results are shown in FIG. 25. Precipitation start was observed after about 10 minutes of FaSSIF addition. The pH after the addition of FaSSIF was 5.8.

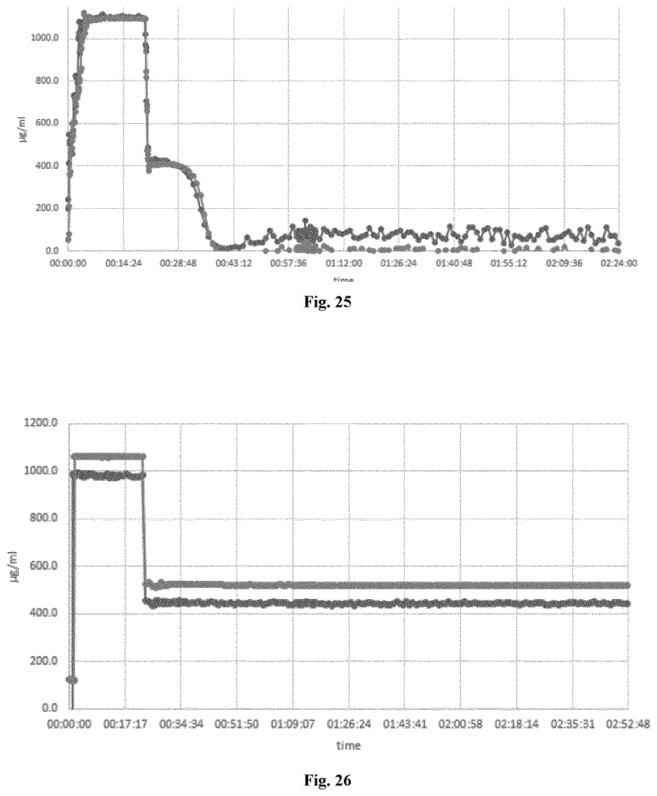

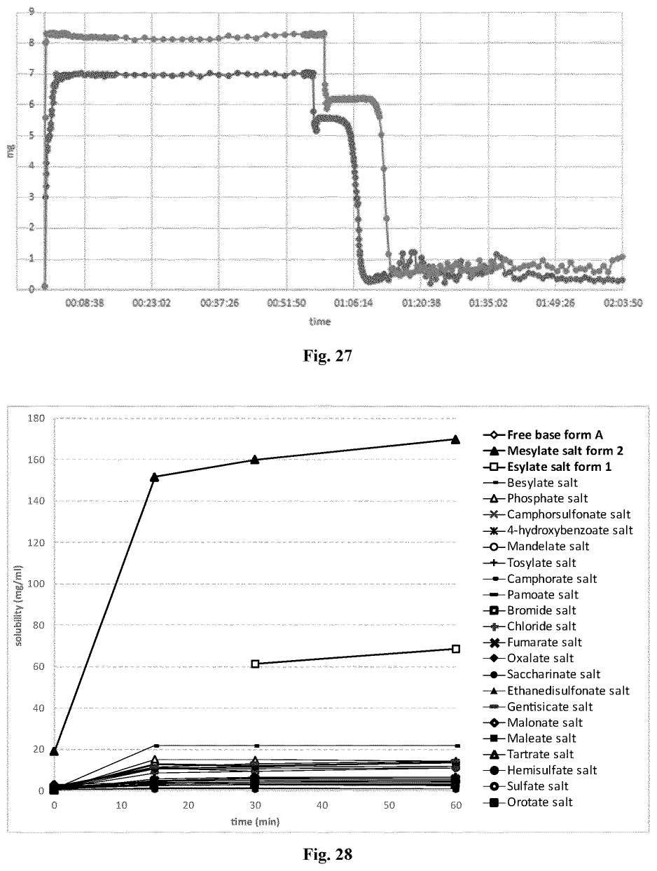

Mesylate salt Form 2 of compound A was tested in duplicate and the results are shown in FIG. 26. No precipitation was observed after the FaSSIF addition. The pH measured at the end of the experiment was 4.5.

A second experiment was conducted, with an additional step of adjusting the pH to 6.5 after FaSSIF addition. Both free base Form A and mesylate salt Form 2 of compound A were tested.

The results are shown in FIG. 27. Surprisingly, the mesylate salt Form 2 of compound A showed a longer duration of the parachute effect than the free base Form A of compound A, with an estimated supersaturation duration of 14 minutes versus 8 minutes shown by the free base Form A. This is indicative of the mesylate salt Form 2 of compound A having a higher chance to increase in vivo bioavailability than free base Form A of compound A. It can be appreciated that this potential improvement in absorption profile makes mesylate salt Form 2 of compound A particularly suitable for oral administration.

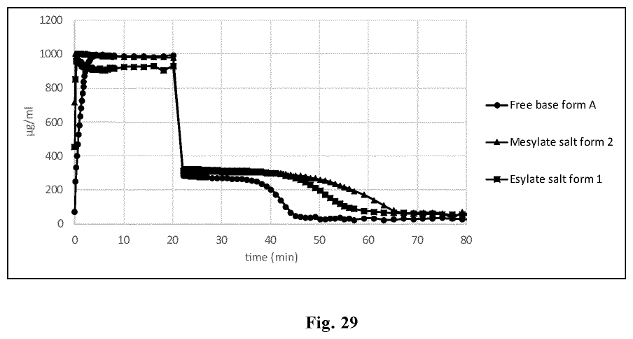

A third experiment was conducted on esylate salt Form 1 of compound A, using the same protocol of the second experiment, with free base Form A and mesylate salt Form 2 of compound A as controls. The experiment was aimed at evaluating comparatively a possible parachute effect of the esylate salt and its duration. Precipitation of the free base was observed after roughly 20 minutes, while precipitation of the esylate salt and mesylate salt were observed after 30 and 40 minutes respectively. The results are shown in FIG. 29. Esylate salt Form 1 and mesylate salt Form 2 of compound A showed a longer duration of the parachute effect compared to free base Form A, indicative of a potentially improved absorption profile.

### Example 11: Formulations

The suitability in formulation of mesylate salt Form 2 of Compound A was tested in comparison with the free base Form A.

The drug content and homogeneity of the mesylate salt Form 2 and the free base Form A of Compound A in 20% w/v SBE-PCD (Sulfobutyl-ether-β-Cyclodextrin, Captisol; CYDEX Pharmaceutical) were tested at different citrate buffer concentrations such as 0.05 M, 0.1 M, 0.5 M and 1 M, at pH 2.5. To prepare the vehicle, the proper amount of citric acid (Sigma Aldrich; CAS: 77-92-9) was dissolved in water in a volumetric flask, then the pH was increased up to 2.5 using 1M NaOH. Then water was added and brought to the final volume. SBE-PCD was weighed and added in a beaker containing the citrate buffer, then left under stirring until it was dissolved. The required volume of vehicle was added to a known amount of Compound A free base Form A or Mesylate salt Form 2, then sonicated at RT for at least 10 min. The formulation was kept under magnetic stirring for at least 10 min before analysis. The stability in terms of content uniformity and pH were evaluated for up to 8 days. The samples were kept at RT and the nominal concentration was 40 mg/ml. The results are reported in Tables 12 and 13.

TABLE 12
Mesylate salt Form 2 of compound A Formulations

| 20% SBE-βCD | Citrate buffer (M) | pH | Appearance |
|---|---|---|---|
| ✓ | 0.05 | 3.0 | Solution |
| ✓ | 0.1 | 3.0 | Solution |
| ✓ | 0.5 | 2.8 | Solution |
| ✓ | 1 | 2.8 | Solution |

TABLE 13
Free base Form A of compound A Formulations

| 20% SBE-βCD | Citrate Buffer (M) | pH | Appearance |
|---|---|---|---|
| ✓ | 0.05 | 3.1 | Fine suspension |
| ✓ | 0.1 | 3.5 | Suspension |
| ✓ | 0.5 | 3.2 | Thick suspension |
| ✓ | 1 | 3.2 | Solution, gel after 24 hours |

As shown in the table 13, the free base Form A of compound A appeared as a suspension in the formulations with a citrate buffer concentration of 0.05 M, 0.1 M and 0.5 M. Free base Form A of compound A appeared as a solution only in the formulation obtained with a citrate buffer concentration of 1 M, however gelification occurred within 24 hours.

The table 12 shows the results with the mesylate salt Form 2. Stable solutions were obtained with all concentrations of citrate buffer. No gelification was observed during the test period, differently from the formulations with free base Form A with 1 M citrate buffer that gelified at 24 hours.

It can be appreciated that mesylate salt Form 2 of compound A has a better formulability in solution than free base Form A of compound A.

The formulation having a 0.05 M citrate buffer concentration was chosen to be tested with the addition of polymers to investigate the effect due to a potential viscosity increase.

Different percentage of polymers were used, e.g. 5% w/v of polyvinylpyrrolidone (PVP) 40 or 1% w/v HPMC.

The formulations were studied with or without the use of the surfactant D-α-tocopherol polyethylene glycol succinate (Vit. E TPGS, Sigma Aldrich; CAS: 9002-96-4) in 0.05 M citrate buffer at pH 2.5 at the nominal concentration of 40 mg/ml. To prepare the vehicle the proper amount of citric acid was dissolved in water in a volumetric flask, then the pH was increased up to 2.5 using 0.1 M NaOH. Then water was added and brought to the final volume. The polymers PVP 40 (Sigma Aldrich; CAS: 9003-39-8) or HPMC (viscosity 46-60 cP; Sigma Aldrich; CAS: 9004-65-3) were gently dissolved in citrate buffer and left under magnetic stirring for at least 1 hour to their complete hydration. When required, Vit. E TPGS was subsequently added to the media and gently left under magnetic stirring to avoid bubbles formation.

The required volume of vehicle was added to a known amount of Compound A free base Form A or Mesylate salt Form 2, then sonicated at RT for at least 10 min. The formulation was kept under magnetic stirring for at least 10 min before analysis.

The results are shown in Table 14.

TABLE 14
Formulations with polymer

| | | Found | Mean | |
|---|---|---|---|---|
| | | conc. | recovery | |
| Vehicles | Compound A | (mg/mL) | (%) | Appearance |
| 1% w/v HPMC | Free base | 41.96 | 104.9 | Suspension |
| 1% w/v Vit. | Form A | | | |
| E TPGS | | | | |
| 5% w/v PVP | Free base | 40.12 | 100.3 | Suspension |
| | Form A | | | |
| 5% w/v PVP | Free base | 44.47 | 111.18 | Suspension |
| 1% w/v Vit. | Form A | | | |
| E TPGS | | | | |
| 1% w/v HPMC | Mesylate salt | 44.44 | 111.10 | Solution |
| 1% w/v Vit. | Form 2 | | | |
| E TPGS | | | | |
| 5% w/v PVP | Mesylate salt | 46.51 | 116.30 | Solution |
| | Form 2 | | | |
| 5% w/v PVP | Mesylate salt | 44.87 | 112.20 | Solution |
| 1% w/v Vit. | Form 2 | | | |
| E TPGS | | | | |

As seen in table 14, all the formulations with the free base Form A appeared as suspensions.

Even increasing the viscosity by adding polymers, a better formulability in solution was obtained with the mesylate salt Form 2 for all the excipients considered.

### Example 12: Two-Sector Dissolution Studies—Formulations

The formulation comprising the mesylate salt Form 2 of compound A of Example 11 (Formulation B) was tested in a two-sector dissolution study according to Example 10 in comparison with a formulation comprising the free base Form A (Formulation A).

The study was performed with formulations at 40 mg/ml.

The formulations tested with their composition and properties are reported below in Table 15.

TABLE 15
Formulations compositions

| | | | Citrate | | | |
|---|---|---|---|---|---|---|
| | | | buffer | | | Super |
| | | | pH 2.5 | | | saturation |
| Form. | Compound A | Excipients | conc. (M) | pH | Appearance | time (min) |
| A | Free base | 1% w/v HPMC | 0.05M | 3.1 | Suspension | 16 |
| | Form A | High viscosity | | | | |
| | | 1% w/v Vit. E | | | | |
| | | TPGS | | | | |
| B | Mesylate | 1% w/v HPMC | 0.05M | 2.5 | Solution | 31 |
| | salt | High viscosity | | | | |
| | Form 2 | 1% w/v Vit. E | | | | |
| | | TPGS | | | | |

According to the present invention, the supersaturation duration higher than 20 minutes can be considered sufficient to identify the formulations with a better chance to increase in vivo bioavailability, as this phenomena predicts an higher dissolved amount available for absorption for a longer time.

As can be appreciated in Table 15, the formulation B comprising the mesylate salt Form 2 shows a supersaturation measured at 31 minutes against the 16 minutes of the formulation A, clearly demonstrating the advantageous likelihood of an increased in vivo bioavailability.

1: A salt of (R)-6-(5-fluoropyridin-2-yl)-8-methoxy-N-(1-(5-methyl-1,2,4-oxadiazol-3-yl) ethyl)quinazolin-4-amine selected from the group consisting of a mesylate salt and an esylate salt.

2: The salt according to claim 1, which is a crystalline mesylate salt of hemihydrate form.

3: The salt according to claim 1, which is a crystalline mesylate salt Form 2 characterized by an X-ray powder diffraction pattern comprising characteristic peaks at 6.6, 11.8 and 19.2±0.2 degrees 2-theta.

4: The salt according to claim 1, which is a crystalline mesylate salt Form 2 characterized by an X-ray powder diffraction pattern comprising characteristic peaks at 6.6, 9.3, 11.8, 19.2 and 27.4±0.2 degrees 2-theta.

5: The salt according to claim 1, which is a crystalline mesylate salt Form 2 characterized by an X-ray powder diffraction pattern comprising characteristic peaks at 6.6, 9.3, 11.8, 18.1, 18.5, 19.2, 20.0, 22.4, 25.7, and 27.4±0.2 degrees 2-theta.

6: The salt according to claim 1, which is a crystalline esylate salt Form 1 characterized by an X-ray powder diffraction pattern comprising characteristic peaks at 5.8, 16.3 and 17.7±0.2 degrees 2-theta.

7: The salt according to claim 1, which is a crystalline esylate salt Form 1 characterized by an X-ray powder diffraction pattern comprising characteristic peaks at 5.8, 8.7, 16.3, 17.7 and 20.1±0.2 degrees 2-theta.

8: The salt according to claim 1, which is a crystalline esylate salt Form 1 characterized by an X-ray powder diffraction pattern comprising characteristic peaks at 5.8, 8.7, 16.3, 17.7, 20.1, 21.5, 22.3, 23.5, 25.1 and 29.1±0.2 degrees 2-theta.

9. (canceled)

10: A pharmaceutical composition comprising the salt according to claim 1, either alone or in combination with another one or more active ingredients, in admixture with one or more pharmaceutically acceptable carriers or excipients.

11: The pharmaceutical composition according to claim 10, formulated for oral administration.

12. (canceled)

13. A method for treating a disease wherein a P2X₃ receptor is involved, comprising administering the salt according to claim 1 to a subject in need thereof.

14. A method for treating a respiratory disease selected from the group consisting of cough, sub-acute or chronic cough, treatment-resistant cough, idiopathic chronic cough, post-viral cough, iatrogenic cough, asthma, idiopathic pulmonary fibrosis (IPF), chronic obstructive pulmonary disease (COPD), cough associated with respiratory disease, asthma and bronchospasm, comprising administering the salt according to claim 1 to a subject in need thereof.

15: A process for the preparation of a crystalline salt of (R)-6-(5-fluoropyridin-2-yl)-8-methoxy-N-(1-(5-methyl-1,2,4-oxadiazol-3-yl)ethyl)quinazolin-4-amine (compound A) selected from the group consisting of a mesylate salt and an esylate salt, comprising:
1) suspending compound A free base in a polar solvent or solvent mixture;
2) adding methanesulfonic acid or ethanesulfonic acid; and
3) isolating a resulting crystalline salt by filtration.

16: The process according to claim 15, wherein the polar solvent or solvent mixture comprises at least one selected from the group consisting of acetone, tetrahydrofuran, isopropanol, and water.

17: The process according to claim 16, comprising:
1) suspending compound A free base in a polar solvent or solvent mixture;
2) adding methanesulfonic acid;
2a) drying a solution obtained in 2);
2b) suspending a solid obtained in 2a) in a polar solvent or solvent mixture and adding an antisolvent thus obtaining precipitation of a crystalline salt; and
3) isolating a resulting crystalline compound A mesylate salt by filtration.

18: The process according to claim 17, wherein in 1) and 2b) the polar solvent or solvent mixture comprises at least one selected from the group consisting of acetone, tetrahydrofuran, isopropanol, and water, and in 2b) the antisolvent is heptane.

19: The process according to claim 15, further comprising washing the filtered crystalline mesylate salt or esylate salt with one or more polar solvents or solvent mixture.

20: The process according to claim 15, further comprising recrystallizing the crystalline mesylate salt or esylate salt with a polar solvent or solvent mixture.

21: The process according to claim 15, further comprising recrystallizing the crystalline mesylate salt of compound A from a mixture of isopropanol and water.

22: The process according to claim 15, wherein the salt is crystalline mesylate salt Form 2.

23: The process according to claim 15, wherein the salt is crystalline esylate salt Form 1.
